(19) United States
(12) Patent Application Publication
(10) Pub. No.: US 2019/0254287 A1
(43) Pub. Date: Aug. 22, 2019

(54) Compositions Comprising Bacillus Licheniformis and Bacillus Subtilis and Methods of Use For Controlling Nematodes

(71) Applicant: FMC Corporation, Philadelphia, PA (US)

(72) Inventors: Rodrigo T. Marques, Campinas (BR); Giuliano Pauli, Piracicaba (BR); Vincent J. Spadafora, Yardley, PA (US); Matthew F. Pye, Newton, PA (US); Guozhi Wang, Oakland Gardens, NY (US)

(21) Appl. No.: 15/563,084

(22) PCT Filed: Aug. 30, 2017
(86) PCT No.: PCT/US2017/049429

§ 371 Date: Sep. 29, 2017

Related U.S. Application Data
(60) Provisional application No. 62/381,860, filed on Aug. 31, 2016.
(60) Provisional application No. 62/381,814, filed on Aug. 31, 2016.

(51) Int. Cl. A01N 63/02; A01N 25/04; A01N 25/10; C12N 1/20; A01N 25/14

(52) U.S. Cl. CPC A01N 63/02; A01N 25/04; A01N 25/14; C12N 1/20; A01N 25/10

(57) ABSTRACT

A composition is provided containing active ingredients Bacillus licheniformis CH200 and Bacillus subtilis CH201 for plant seed treatment or in-furrow application to control plant pathogenic nematodes. The composition is efficacious at suppressing nematodes, including Meloidogyne spp. and Pratylenchus. Related articles of manufacture and methods are also provided.

### CROSS REFERENCE TO RELATED APPLICATIONS

This application claims the benefit of U.S. provisional application No. 62/381,814, filed Aug. 31, 2016, and U.S. provisional application No. 62/381,860, filed Aug. 31, 2016, the disclosures of which are hereby incorporated herein by reference in their entireties.

### TECHNICAL FIELD

The presently disclosed subject matter relates to wettable powder compositions containing active ingredients Bacillus licheniformis CH200 and Bacillus subtilis CH201 for seed treatment or in-furrow application to control plant pathogenic nematodes.

### BACKGROUND

A number of microorganisms having beneficial effects on plant growth and health are known to be present in the soil, to live in association with plants specifically in the root zone (Plant Growth Promoting Rhizobacteria “PGPR”), or to reside as endophytes within the plant. Their beneficial plant growth promoting properties include nitrogen fixation, iron chelation, phosphate solubilization, inhibition of non-beneficial microrganisms, resistance to pests, Induced Systemic Resistance (ISR), Systemic Acquired Resistance (SAR), decomposition of plant material in soil to increase useful soil organic matter, and synthesis of phytohormones such as indole-acetic acid (IAA), acetoin and 2,3-butanediol that stimulate plant growth, development and responses to environmental stresses such as drought. In addition, these microorganisms can interfere with a plant's ethylene stress response by breaking down the precursor molecule, 1-aminocyclopropane-1-carboxylate (ACC), thereby stimulating plant growth and slowing fruit ripening. These beneficial microorganisms can improve soil quality, plant growth, yield, and quality of crops. Various microorganisms exhibit biological activity such as to be useful to control plant diseases. Such biopesticides (living organisms and the compounds naturally produced by these organisms) are safer and more biodegradable than synthetic fertilizers and pesticides.

Some members of the species Bacillus have been reported as biocontrol strains, and some have been applied in commercial products (Joseph W. Kloepper, et al. 2004, Phytopathology Vol. 94, No. 11, 1259-1266). For example, strains currently being used in commercial biocontrol products include: Bacillus pumilus strain QST2808, used as active ingredient in SONATA® and BALLAD®-PLUS, produced by BAYER CROP SCIENCE; Bacillus pumilus strain GB34, used as active ingredient in YIELDSHIELD, produced by BAYER CROP SCIENCE; Bacillus subtilis strain QST713, used as the active ingredient of SERENADE′, produced by BAYER CROP SCIENCE; Bacillus subtilis strain GBO3, used as the active ingredient in KODIAK® and SYSTEM3®, produced by HELENA CHEMICAL COMPANY. Various strains of Bacillus thuringiensis and Bacillus firmus have been applied as biocontrol agents against nematodes and vector insects and these strains serve as the basis of numerous commercially available biocontrol products, including NORTICA® and PONCHO™-VOTIVO™, produced by BAYER CROP SCIENCE. In addition, Bacillus strains currently being used in commercial biostimulant products include: Bacillus amyloliquefaciens strain FZB42 used as the active ingredient in RHIZOVITAL® 42, produced by ABiTEP GmbH, as well as various other Bacillus subtilus species that are included as whole cells including their fermentation extract in biostimulant products, such as FULZYME produced by JHBiotech Inc.

The presently disclosed subject matter provides improved compositions of Bacillus licheniformis CH200 and Bacillus subtilis CH201 for plant seed treatment or in-furrow application to control plant pathogenic nematodes and benefit plant growth and yield.

### SUMMARY

In one embodiment, a wettable powder composition is provided for plant seed treatment or in-furrow application to control plant pathogenic nematodes and to benefit plant growth and yield, the composition comprising by weight percent: 5-20% of a biologically pure culture of not less than about 1×10¹¹ CFU/g Bacillus licheniformis CH200 deposited as DSM 17236, or a mutant thereof having all the identifying characteristics thereof; 5-20% of a biologically pure culture of not less than about 1×10¹¹ CFU/g Bacillus subtilis CH201 deposited as DSM 17231, or a mutant thereof having all the identifying characteristics thereof; 5-15% maltodextrin; 35-45% calcium carbonate; and 5-15% silica.

In one embodiment, a wettable powder composition is provided for plant seed treatment or in-furrow application to control plant pathogenic nematodes and to benefit plant growth and yield, the composition comprising by weight percent: 20% of a biologically pure culture of not less than about 1×10¹¹ CFU/g Bacillus licheniformis CH200 deposited as DSM 17236, or a mutant thereof having all the identifying characteristics thereof; 20% of a biologically pure culture of not less than about 1×10¹¹ CFU/g Bacillus subtilis CH201 deposited as DSM 17231, or a mutant thereof having all the identifying characteristics thereof; 10% maltodextrin; 40% calcium carbonate; and 10% silica.

In one embodiment, an article of manufacture is provided comprising: a plant seed treated with a composition for controlling plant pathogenic nematodes and benefiting plant growth and yield, the composition comprising: 5-20% of a biologically pure culture of not less than about 1×10¹¹ CFU/g Bacillus licheniformis CH200 deposited as DSM 17236, or a mutant thereof having all the identifying characteristics thereof; 5-20% of a biologically pure culture of not less than about 1×10¹¹ CFU/g Bacillus subtilis CH201 deposited as DSM 17231, or a mutant thereof having all the identifying characteristics thereof; 5-15% maltodextrin; 35-45% calcium carbonate; and 5-15% silica.

In one embodiment, an article of manufacture is provided comprising: a plant seed treated with a composition for controlling plant pathogenic nematodes and benefiting plant growth and yield, the composition comprising: 20% of a biologically pure culture of not less than about 1×10¹¹ CFU/g Bacillus licheniformis CH200 deposited as DSM 17236, or a mutant thereof having all the identifying characteristics thereof; 20% of a biologically pure culture of not less than about 1×10¹¹ CFU/g Bacillus subtilis CH201 deposited as DSM 17231, or a mutant thereof having all the identifying characteristics thereof; 10% maltodextrin; 40% calcium carbonate; and 10% silica.

In one embodiment, a method is provided for controlling plant pathogenic nematodes and benefiting plant growth and yield of a plant seed, the method comprising: applying a composition to a plant seed, wherein the composition comprises by weight percent: 5-20% of a biologically pure culture of not less than about 1×10¹¹ CFU/g Bacillus licheniformis CH200 deposited as DSM 17236, or a mutant thereof having all the identifying characteristics thereof; 5-20% of a biologically pure culture of not less than about 1×10¹¹ CFU/g Bacillus subtilis CH201 deposited as DSM 17231, or a mutant thereof having all the identifying characteristics thereof; 5-15% maltodextrin; 35-45% calcium carbonate; and 5-15% silica, wherein the composition applied to the seed improves plant growth and/or yield in the presence of a plant pathogenic nematode, improves plant resistance to a plant pathogenic nematode, or reduces plant infection by a plant pathogenic nematode, and combinations thereof.

In one embodiment, a method is provided for controlling plant pathogenic nematodes and improving growth and yield of a plant, the method comprising: delivering to seed of a plant, roots of a plant, or soil surrounding a plant a composition comprising by weight percent: 5-20% of a biologically pure culture of not less than about 1×10¹¹ CFU/g Bacillus licheniformis CH200 deposited as DSM 17236, or a mutant thereof having all the identifying characteristics thereof; 5-20% of a biologically pure culture of not less than about 1×10¹¹ CFU/g Bacillus subtilis CH201 deposited as DSM 17231, or a mutant thereof having all the identifying characteristics thereof; 5-15% maltodextrin; 35-45% calcium carbonate; and 5-15% silica, wherein the composition delivered to seed of the plant, roots of the plant, or soil surrounding the plant improves the plant growth and/or yield in the presence of a plant pathogenic nematode, improves the plant resistance to a plant pathogenic nematode, or reduces the plant infection by a plant pathogenic nematode, and combinations thereof.

In one embodiment, a process is provided for preparing a composition as shown in Table 1, the process comprising mixing, in a dry or powder form: a biologically pure culture of not less than about 1×10¹¹ CFU/g Bacillus licheniformis CH200 deposited as DSM 17236, or a mutant thereof having all the identifying characteristics thereof; a biologically pure culture of not less than about 1×10¹¹ CFU/g Bacillus subtilis CH201 deposited as DSM 17231, or a mutant thereof having all the identifying characteristics thereof; a maltodextrin; a calcium carbonate; and a silica, wherein the mixing is in a ratio to effect the weight percent of the composition according to Table 1.

In one embodiment, a process is provided for preparing a composition, the process comprising mixing, in a dry or powder form: a biologically pure culture of not less than about 1×10¹¹ CFU/g Bacillus licheniformis CH200 deposited as DSM 17236, or a mutant thereof having all the identifying characteristics thereof; a biologically pure culture of not less than about 1×10¹¹ CFU/g Bacillus subtilis CH201 deposited as DSM 17231, or a mutant thereof having all the identifying characteristics thereof; a maltodextrin; a calcium carbonate; and a silica, wherein the mixing is in a ratio to effect the weight percent of the composition consisting of 5-20% of the culture deposited as DSM 17236, 5-20% of the culture deposited as DSM 17231, 5-15% maltodextrin, 35-45% calcium carbonate, and 5-15% silica.

In one embodiment, a composition is provided for plant seed treatment or in-furrow application to control plant pathogenic nematodes, wherein the composition is in the form of a dust, a dry wettable powder, a spreadable granule, or a dry wettable granule, the composition comprising by weight percent: −40% of a culture of not less than about 1×10¹¹ CFU/g of Bacillus licheniformis CH200 deposited as DSM 17236, or a mutant thereof having all the identifying characteristics thereof; 7-40% of a culture of not less than about 1×10¹¹ CFU/g of Bacillus subtilis CH201 deposited as DSM 17231, or a mutant thereof having all the identifying characteristics thereof.

### BRIEF DESCRIPTION OF THE DRAWINGS

FIG. 1 is a graph showing percent adhesion to soybean seed versus concentration of maltodextrin according to one or more embodiments of the present invention.

FIG. 2 is a schematic drawing showing the method of estimating the percent adhesion to soybean seed with the varying concentrations of maltodextrin as shown in FIG. 1, with the powder formulation shown in (A); after liquid addition to the powder formulation shown in (B); and after mixing of soybean seed with the liquid formulation shown in (C).

FIG. 3 is a schematic drawing showing the results of the seed treatment testing of Formulations T1 (A & B), T2 (C & D), and T3 (E & F) according to one or more embodiments of the present invention.

### DETAILED DESCRIPTION

The presently disclosed subject matter now will be described more fully hereinafter with reference to the accompanying Figures, in which some, but not all embodiments of the presently disclosed subject matter are shown. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

Following long-standing patent law convention, the terms “a,” “an,” and “the” refer to “one or more” when used in this application, including the claims. Thus, for example, reference to “a plant” includes a plurality of plants, unless the context clearly is to the contrary, and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

In one embodiment, the phrases “a biologically pure culture of a Bacillus licheniformis CH200” and “a biologically pure culture of a Bacillus subtilis CH201” each individually refer to one or a combination of: spores of a biologically pure fermentation culture of the bacterial strain, vegetative cells of a biologically pure fermentation culture of the bacterial strain, one or more products of a biologically pure fermentation culture of the bacterial strain, a culture solid of a biologically pure fermentation culture of the bacterial strain, a culture supernatant of a biologically pure fermentation culture of the bacterial strain, and an extract of a biologically pure fermentation culture of the bacterial strain.

In another embodiment, the phrases “a biologically pure culture of a Bacillus licheniformis CH200” and “a biologically pure culture of a Bacillus subtilis CH201” each individually refer to one or a combination of: spores of a biologically pure fermentation culture of the bacterial strain, vegetative cells of a biologically pure fermentation culture of the bacterial strain, one or more products of a biologically pure fermentation culture of the bacterial strain, and a culture solid of a biologically pure fermentation culture of the bacterial strain. In one variant of this embodiment, each phrase may refer to the spores of a biologically pure fermentation culture of the bacterial strain.

In still another embodiment, the phrases “a biologically pure culture of a Bacillus licheniformis CH200” and “a biologically pure culture of a Bacillus subtilis CH201” each individually refer to one or a combination of: a culture supernatant of a biologically pure fermentation culture of the bacterial strain, and an extract of a biologically pure fermentation culture of the bacterial strain.

In embodiments, compositions are provided containing active ingredients Bacillus licheniformis CH200 and Bacillus subtilis CH201 for plant seed treatment or in-furrow application to control plant pathogenic nematodes. The compositions may be formulated to provide for seed adhesion, solubility, suspensibility, and/or microorganism stability.

In one or more embodiments of the present invention, a wettable powder composition is provided containing active ingredients Bacillus licheniformis CH200 and Bacillus subtilis CH201 for plant seed treatment or in-furrow application to control plant pathogenic nematodes and benefit plant growth and yield. The biological-based wettable powder composition provides protection from key plant pathogenic nematodes, with superior seed treatment properties and also suitability for use as an in-furrow application. The in-furrow application of the composition can be used alone or as a complement to chemical and/or biological seed treatments. The properties of the wettable powder composition include root enhancement and direct nematicide effect against nematode eclosion. In addition to the nematicidal properties, the wettable powder composition of the invention supports faster crop development with a stronger root system and higher yields.

The wettable powder composition of the invention provides for both optimal seed adhesion and flowability properties, and also provides solubility when applied in-furrow. The composition achieves the seed adhesion properties without compromising solubility for the in-furrow application. An additional benefit of the compositions of the invention is an enhanced shelf-life for the microorganisms by absorbing residual water. The composition is provided as a robust formulation as both a WS (Wettable for Seed Treatment) or WP (Wetable Powder) for in-furrow and seed treatment applications.

In accordance with one aspect of the present invention, a combination of ingredients has been discovered that provides a formulation that is physically stable, and preserves the shelf life of the microorganisms, while also providing seed adhesion and flowability properties without compromising solubility/wettablility when applied in-furrow. Development of the wettable composition of the invention is described herein at EXAMPLE 1. The wettable powder of the invention contains as active ingredients the microorganisms Bacillus licheniformis CH200 and Bacillus subtilis CH201 at a concentration to provide optimal control of plant pathogenic nematodes and to promote plant growth and yield. The strain of Bacillus licheniformis CH200 was deposited on Apr. 7, 2005 at Deutsche Sammlung von Mikroorganismen und Zellkulturen GmbH, Mascheroder Weg 1 b, D-38124 Braunschweig (DSMZ) and given the accession No. DSM 17236. The strain of Bacillus subtilis CH201 was deposited on Apr. 7, 2005 at Deutsche Sammlung von Mikroorganismen und Zellkulturen GmbH, Mascheroder Weg 1 b, D-38124 10 Braunschweig (DSMZ) and given the accession No. DSM 17231.

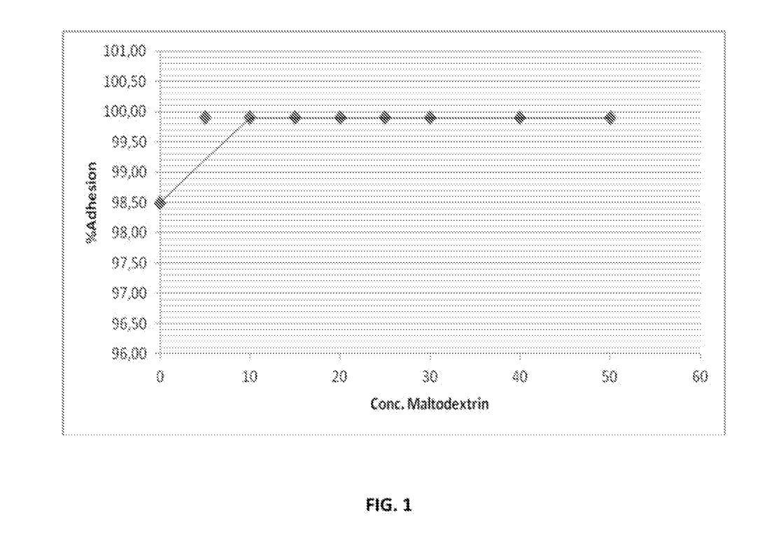

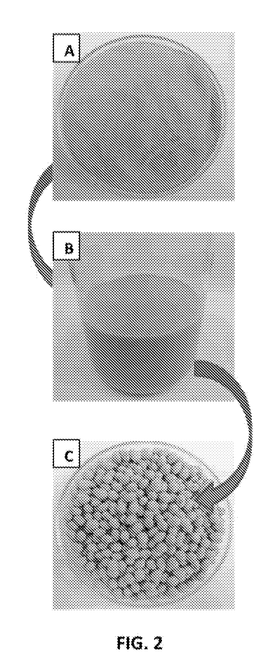

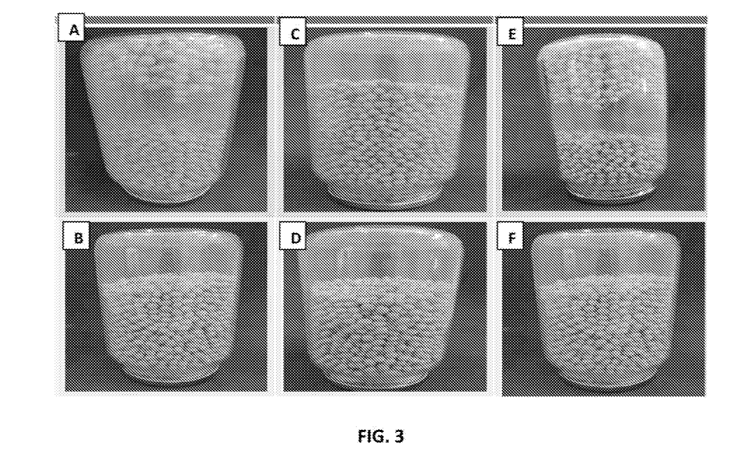

In addition to the active biological ingredients, the wettable powder composition of the invention contains the following ingredients: calcium carbonate, maltodextrin, and silica, which were optimized at a concentration to provide seed adhesion and flowability properties without compromising solubility/wettablility when applied in-furrow (see EXAMPLE 1). The optimal amount of maltodextrin was chosen based on the studies of concentration versus soybean seed adhesion as shown in FIG. 1 and FIG. 2. The percentage of adhesion was determined based on what remained on the seed without consideration of what remained in the jar. Based on the results, the optimal amount of maltodextrin was determined to be 5-15%. Various formulations were produced for seed treatment testing by homogenizing the ingredients in powder form. It was discovered that simple mixing of the ingredients by homogenization was preferable to any form of grinding. Three formulations were tested (Formulations T1, T2, and T3 shown in EXAMPLE 1). The results of the seed treatment testing are shown in FIG. 3. It is clear from the test results that Formulation T2 (FIGS. 3C & 3D) has the superior seed treatment properties.

Thus, one embodiment of the wettable powder composition of the invention is the Formulation T2 shown below in Table 1. The manufacture of this composition includes addition of the following ingredients in powder form in the order specified: 1) maltodextrin; 2) calcium carbonate; 3) silicon dioxide; 4) CH200; and 5) CH201, with a homogenization time of about 5 min.

TABLE 1
Composition in powder form for seed treatment or in-furrow application
referred to herein as “T2.”

| Ingredients | W/W % |
|---|---|
| Bacillus licheniformis CH200 | 20 |
| Bacillus subtilis CH201 | 20 |
| Maltodextrin | 10 |
| Calcium carbonate | 40 |
| Silica | 10 |

In one embodiment, a process is provided for preparing a composition as shown in Table 1, the process comprising mixing, in a dry or powder form: a biologically pure culture of not less than about 1×10¹¹ CFU/g Bacillus licheniformis CH200 deposited as DSM 17236, or a mutant thereof having all the identifying characteristics thereof; a biologically pure culture of not less than about 1×10¹¹ CFU/g Bacillus subtilis CH201 deposited as DSM 17231, or a mutant thereof having all the identifying characteristics thereof; a maltodextrin; a calcium carbonate; and a silica, where the mixing is in a ratio to effect the weight percent of the composition according to Table 1.

In another embodiment, a process is provided for preparing a composition, the process comprising mixing, in a dry or powder form: a biologically pure culture of not less than about 1×10¹¹ CFU/g Bacillus licheniformis CH200 deposited as DSM 17236, or a mutant thereof having all the identifying characteristics thereof; a biologically pure culture of not less than about 1×10¹¹ CFU/g Bacillus subtilis CH201 deposited as DSM 17231, or a mutant thereof having all the identifying characteristics thereof; a maltodextrin; a calcium carbonate; and a silica, wherein the mixing is in a ratio to effect the weight percent of the composition consisting of 5-20% of the culture deposited as DSM 17236, 5-20% of the culture deposited as DSM 17231, 5-15% maltodextrin, 35-45% calcium carbonate, and 5-15% silica.

The beneficial activity of the formulated composition of the CH200 and CH201 bacterial strains provided herein for controlling plant pathogenic nematodes and increasing plant growth and yield is exemplified by the studies described herein at EXAMPLES 2-4. In these studies the wettable powder composition shown in Table 1 (“T2”) was used as a seed treatment in field trials on soybean, cotton, corn, and dry bean. The T2 composition was prepared as described in EXAMPLE 1, and the final concentration of each of the CH200 and CH201 strains in the T2 composition is not less than about 1×10¹¹ CFU/g.

EXAMPLE 2 describes field trials of seed treatment with the T2 composition for soybean, corn, cotton and dry bean seeds. In the trials, all soybean, corn, and dry bean seeds were treated with commercially available product MAXIM® XL (SYNGENTA, INC) containing fungicidal active ingredients Fludioxonil and Metalaxyl-M, which is referred to herein as “Check”, and all cotton seeds were treated with commercial products DEROSAL PLUS (BAYER CROPSCIENCE, INC) containing active ingredient Carbendazim, similarly referred to herein as “Check”. In addition, a commercially available biofungicide product QUALITY (Laboratórios Biocontrole Farroupilha) containing active ingredient Thrichoderma and a commercially available nematicidal product AVICTA® (SYNGENTA, INC) containing active ingredient Abamectin were included in the studies in soy, cotton, and corn. In the case of the dry bean studies, a commercial nematicide FURADAN (FMC Corporation) containing active ingredient Carbofuran was included instead of AVICTA®. The field trials were performed in Brazil (2-6 separate trials were performed for each of the crops) under natural disease pressure conditions that included low to high nematode pressure.

The results of six separate soybean field trials performed under a range of low, medium, and high nematode pressure are shown in Table 3 (standability, seedling height, root length, and yield). Specifically, for standability, treatment of seed with the T2 formulation of Bacillus licheniformis CH200 and Bacillus subtilis CH201 at 70 g/100 kg and 100 g/100 kg seed provided at least about a 5% improvement over the commercial products QUALITY and AVICTA®. For seedling development, treatment of seed with the T2 formulation of CH200 and CH201 strains at all rates resulted in an increase in height as compared to the check and resulted in a 9.9% increase in height as compared to the check at the highest rate. Further, the T2 formulation of CH200 and CH201 strains provided comparable results to QUALITY, and at the highest two rates outperformed AVICTA® by at least a 2.4% increase in height. For root mass development, the T2 formulation of CH200 and CH201 strains outperformed the check at all rates by at least 12.6 percent. Further, for the two highest rates, the T2 formulation of CH200 and CH201 strains outperformed both QUALITY and AVICTA in root mass development by 4.2% and 2.6%, respectively. For yield, the T2 formulation of CH200 and CH201 strains outperformed the check at all rates by at least 8% percent and for the highest rate increased yield by 15.1%. All rates of the T2 formulation of CH200 and CH201 strains promoted higher yields than QUALITY. Further, the highest rate of the T2 formulation of CH200 and CH201 strains resulted in an increase in yield over AVICTA® of 3.4%. Thus, soybean seed treatment with the T2 formulation of CH200 and CH201 provides significant improvements over a standard chemical treatment check and commercial nematicides in plant development and yield under a range of low, medium, and high nematode pressure.

The results of six separate cotton field trials performed under a range of low and medium nematode pressure are shown in Table 4 (standability, seedling height, root length, and yield). Specifically, for seedling height, treatment of seed with the T2 formulation of CH200 and CH201 strains at all rates resulted in a statistical increase in height as compared to the check. At the highest application rate, application of the combination of strain provided a 9.3% and 8.7% increase in height as compared to the check and as compared to QUALITY, respectively. Further, at the highest rate, the T2 formulation of CH200 and CH201 strains outperformed AVICTA® by a 2.3% increase in height. For root development, treatment of seed with the T2 formulation of CH200 and CH201 strains at all rates resulted in a statistical increase in length as compared to the check and as compared to AVICTA® (a 9.8% and 2.0% increase in length, respectively, at the highest application rate). Further, treatment with the T2 formulation of CH200 and CH201 strains provided statistically similar results as compared to QUALITY at the highest rate. Treatment with of all rates of the T2 formulation of CH200 and CH201 strains promoted higher yields than the check and both commercial products QUALITY and AVICTA® (12.2%, 5.2%, and 5.0%, respectively, for the highest rate). Thus, cotton seed treatment with the T2 formulation of CH200 and CH201 provides significant improvements over a standard chemical treatment check and commercial nematicides in plant development and yield under low and medium nematode pressure.

The results of four separate corn field trials performed under a range medium and high nematode pressure are shown in Table 5 (seedling height, root length, and yield). For seedling height, treatment of seed with the T2 formulation of CH200 and CH201 strains provided improved results over the commercial products at all rates and, specifically, an improvement of 4.5% over QUALITY and an improvement of 3.2% over AVICTA® at the highest rate. For root development, treatment with the T2 formulation of CH200 and CH201 strains outperformed the check at all rates. At the two highest rates, treatment with the T2 formulation of CH200 and CH201 strains outperformed the check and AVICTA® by at least 14.2% and 5.3%, respectively. Further, at the highest rate, treatment with the T2 formulation of CH200 and CH201 strains performed comparably to QUALITY. For yield, treatment with the T2 formulation of CH200 and CH201 strains outperformed the check at all rates and, specifically, the 100 g/100 kg rate increased yield by 7.9% over the check. In addition, the 100 g/100 kg rate of the T2 formulation of CH200 and CH201 strains provided yields statistically comparable to AVICTA®. Thus, corn seed treatment with the T2 formulation of CH200 and CH201 provides significant improvements in plant development and yield over a standard chemical treatment check and provides comparable improvements in yield to commercial nematicides under medium and high nematode pressure.

The results of two separate dry bean field trials performed under a range of low and high nematode pressure are shown in Table 6 (standability, seedling height, root length, and yield). Specifically, for standability, treatment of seed with the T2 formulation of CH200 and CH201 strains at the highest rate provided a 9.2%, 4.9%, and 11.7% improvement over the check, over QUALITY, and over FURADAN, respectively. For seedling development, treatment of seed with the T2 formulation of CH200 and CH201 strains at all rates resulted in an increase in height as compared to the check and as compared to QUALITY. In addition, the 80 kg/100 kg rate of the the T2 formulation of the CH200 and CH201 strains statistically outperformed FURADAN. For root length, all rates of treatment with the T2 formulation of CH200 and CH201 strains outperformed the check and QUALITY. The 3 lowest treatment rates of the T2 formulation of the CH200 and CH201 strains statistically outperformed or performed comparably to FURADAN. For yield, treatment with the T2 formulation of CH200 and CH201 strains outperformed the check at all rates and for the highest rate increased yield by 18.4%. Treatment with the two highest rates of the T2 formulation of CH200 and CH201 strains promoted higher yields than QUALITY (5.4% and 5.9%, respectively) and promoted comparable yields to FURADAN. Thus, seed treatment with the T2 formulation of CH200 and CH201 provides significant improvements in yield over a standard chemical treatment check and provides comparable to significant improvements in yield as compared to commercial nematicides under low and high nematode pressure.

EXAMPLE 3 describes additional field trials performed in Brazil with soybean seed treated with the formulation of CH200 and CH201 “T2” described in EXAMPLE 1 in areas of high nematode infestation. The results demonstrate that seed treatment with the T2 formulation of CH200 and CH201 strains significantly increases soybean yield, and the increase in yield is greater than that for the commercial product AVICTA® containing chemical active agent Abamectin. In the field trials, all soybean seed was treated with MAXIM® XL containing Fludioxonil and Mefenoxam active chemical fungicidal ingredients and with chemical insecticide active agents Imidacloprid and Bifenthrin according to label (referred to herein as condition (1) base treatment (“BT”)). Condition (2) included further treatment of the seed with T2 at a rate of 70 g/100 kg seed. Condition (3) included further treatment of the seed with T2 at a rate of 100 g/100 kg seed. Condition (4) included further treatment of the seed with commercial product AVICTA® at 100 g/100 kg seed containing chemical active agent Abamectin for controlling nematodes. Field trials were conducted at two different locations with high nematode pressure and the average yield data are shown below in Table 7. The results demonstrate that seed treatment with the T2 formulation of CH200 and CH201 stains significantly increased yield relative to the base treatment at both rates (11.9% and 13.6%, respectively), and outperformed commercial product AVICTA, which provided an increase in yield of 11.0%.

EXAMPLE 4 describes greenhouse studies of soybean demonstrating that application of the T2 formulation of the CH200 and CH201 strains to soybean infected with various types of nematodes can outperform commercially available products based on both chemical and biological active agents. In one experiment, soybean seeds were planted in pots infected with 600 Pratylenchus brachyurus nematodes per pot or 1000-2000 Meloidogyne javanica nematodes per pot. All seed was treated with MAXIM® XL containing Fludioxonil and Mefenoxam active chemical fungicidal ingredients according to label (referred to as condition (1) Base treatment (“BT”)). Condition (2) included further treatment of the seed with T2 at a rate of 100 g/100 kg seed. Condition (3) included further treatment of the seed with commercial product AVICTA at 100 g/100 kg seed containing chemical active agent Abamectin for controlling nematodes. Condition (4) included further treatment of the seed with commercial product NEMAT (SYNGENTA, INC) at 100 g/100 kg seed containing fungal active agent Paecilomyces lilacinus (7.5×10⁹ CFU/g) for controlling nematodes. The data for initial penetration of Meloidogyne javanica nematodes 21 days after inoculation (DAI) are shown in Table 8. The results demonstrate that the T2 formulation of the CH200 and CH201 stains performs statistically as well in controlling nematode penetration as commercial chemical product AVICTA®, and outperforms commercial product NEMAT containing biological active agent Paecilomyces lilacinus. The number of juvenile Meloidogyne javanica nematodes per 5 gram roots at 40 days after the inoculation (DAI) was examined and the results are shown in Table 9. The results demonstrate that the T2 formulation of the CH200 and CH201 stains performs statistically as well in controlling nematode juveniles as commercial chemical product AVICTA, and outperforms commercial product NEMAT containing biological active agent Paecilomyces lilacinus. The number of Meloidogyne javanica nematode eggs per 5 gram roots at 60 DAI was examined and the results are shown in Table 10. The results demonstrate that the T2 formulation of the CH200 and CH201 stains statistically outperforms chemical product AVICTA in controlling nematode eggs on roots. In this case, the T2 formulation of the CH200 and CH201 stains does not perform as well as the commercial product NEMAT containing biological active agent Paecilomyces lilacinus. The P. lilacinus strain is known for its ability to kill nematode eggs. The reproduction factor (RF—Final nematode population divided by Initial nematode population) of Pratylenchus brachyurus nematodes at 60 and 90 DAI was examined and the results are shown in Table 11 below. The results demonstrate that the T2 formulation of the CH200 and CH201 stains statistically outperforms chemical product AVICTA in controlling nematode reproduction at both 60 and 90 days DAI. In addition, the T2 formulation of the CH200 and CH201 stains outperforms the commercial product NEMAT containing biological active agent Paecilomyces lilacinus at 60 DAI and performs as well as this product at 90 DAI. These data show that the T2 formulation of the CH200 and CH201 stains can control nematodes better than the commercial chemical nematicide active agent and can control nematodes as well or even outperform the commercial nematicide product NEMAT containing Paecilomyces lilacinus.

In embodiments, a wettable powder composition is provided for plant seed treatment or in-furrow application to control plant pathogenic nematodes, the composition comprising by weight percent: 5-20% of a biologically pure culture of not less than about 1×10¹¹ CFU/g Bacillus licheniformis CH200 deposited as DSM 17236, or a mutant thereof having all the identifying characteristics thereof; 5-20% of a biologically pure culture of not less than about 1×10¹¹ CFU/g Bacillus subtilis CH201 deposited as DSM 17231, or a mutant thereof having all the identifying characteristics thereof; 5-15% maltodextrin; 35-45% calcium carbonate; and 5-15% silica. In an embodiment, application of the composition benefits plant growth and yield.

In an embodiment, a wettable powder composition is provided for plant seed treatment or in-furrow application to control plant pathogenic nematodes, the composition comprising by weight percent: 20% of a biologically pure culture of not less than about 1×10¹¹ CFU/g Bacillus licheniformis CH200 deposited as DSM 17236, or a mutant thereof having all the identifying characteristics thereof; 20% of a biologically pure culture of not less than about 1×10¹¹ CFU/g Bacillus subtilis CH201 deposited as DSM 17231, or a mutant thereof having all the identifying characteristics thereof; 10% maltodextrin; 40% calcium carbonate; and 10% silica. In an embodiment, application of the composition benefits plant growth and yield.

In an embodiment, a composition for plant seed treatment or in-furrow application to control plant pathogenic nematodes, wherein the composition is in the form of a dust, a dry wettable powder, a spreadable granule, or a dry wettable granule, the composition comprising by weight percent: 7-40% of a culture of not less than about 1×10¹¹ CFU/g of Bacillus licheniformis CH200 deposited as DSM 17236, or a mutant thereof having all the identifying characteristics thereof; 7-40% of a culture of not less than about 1×10¹¹ CFU/g of Bacillus subtilis CH201 deposited as DSM 17231, or a mutant thereof having all the identifying characteristics thereof; wherein the composition comprises a solid carrier. In embodiments, the solid carrier is selected from the group consisting of mono- or di-saccharides, oligo- or poly-saccharides, talc, titanium dioxide, pyrophyllite clay, attapulgite clay, kieselguhr, silica, limestone, bentonite, calcium montmorillonite, sodium, potassium, magnesium, calcium or ammonium salts of acetate, carbonate, chloride, citrate, phosphate, or sulfate, cottonseed husks, wheat flour, soybean flour, pumice, wood flour, ground nut shells, lignin, yeast extracts, fish meal, and mixtures thereof.

In embodiments, the solid carrier comprises maltodextrin, silica, calcium carbonate, or any mixtures thereof. In embodiments, the composition comprises 5-15% of maltodextrin.

In certain embodiments described herein, the plant seed treatment or in furrow application of the composition benefits plant growth and/or yield.

In embodiments, an article of manufacture is provided comprising a plant seed treated with a composition for controlling plant pathogenic nematodes. In certain embodiments, the plant seed comprises soybean, dry bean, cotton, or corn. In an embodiment the composition comprises: 5-20% of a biologically pure culture of not less than about 1×10¹¹ CFU/g Bacillus licheniformis CH200 deposited as DSM 17236, or a mutant thereof having all the identifying characteristics thereof; 5-20% of a biologically pure culture of not less than about 1×10¹¹ CFU/g Bacillus subtilis CH201 deposited as DSM 17231, or a mutant thereof having all the identifying characteristics thereof; 5-15% maltodextrin; 35-45% calcium carbonate; and 5-15% silica. In an embodiment, treatment with the composition benefits plant growth and yield.

In embodiment, an article of manufacture is provided comprising: a plant seed treated with a composition for controlling plant pathogenic nematodes, the composition comprising: 20% of a biologically pure culture of not less than about 1×10¹¹ CFU/g Bacillus licheniformis CH200 deposited as DSM 17236, or a mutant thereof having all the identifying characteristics thereof; 20% of a biologically pure culture of not less than about 1×10¹¹ CFU/g Bacillus subtilis CH201 deposited as DSM 17231, or a mutant thereof having all the identifying characteristics thereof; 10% maltodextrin; 40% calcium carbonate; and 10% silica. In an embodiment, treatment with the composition benefits plant growth and yield.

In embodiments, a method is provided for controlling plant pathogenic nematodes, the method comprising: delivering to seed of a plant, roots of a plant, or soil surrounding a plant a composition described herein. In embodiments, the composition delivered to seed of the plant, roots of the plant, or soil surrounding the plant improves the plant growth and/or yield in the presence of a plant pathogenic nematode, improves the plant resistance to a plant pathogenic nematode, or reduces the plant infection by a plant pathogenic nematode, and combinations thereof.

In embodiments, a method is provided for controlling plant pathogenic nematodes, the method comprising: applying a composition to a plant seed, wherein the composition comprises by weight percent: 5-20% of a biologically pure culture of not less than about 1×10¹¹ CFU/g Bacillus licheniformis CH200 deposited as DSM 17236, or a mutant thereof having all the identifying characteristics thereof; 5-20% of a biologically pure culture of not less than about 1×10¹¹ CFU/g Bacillus subtilis CH201 deposited as DSM 17231, or a mutant thereof having all the identifying characteristics thereof; 5-15% maltodextrin; 35-45% calcium carbonate; and 5-15% silica. In embodiments, the composition applied to the seed improves plant growth and/or yield in the presence of a plant pathogenic nematode, improves plant resistance to a plant pathogenic nematode, or reduces plant infection by a plant pathogenic nematode, and combinations thereof.

In one embodiment of the method for controlling plant pathogenic nematodes by applying the composition of the invention to the plant seed, the composition comprises by weight percent: 20% of a biologically pure culture of not less than about 1×10¹¹ CFU/g Bacillus licheniformis CH200 deposited as DSM 17236, or a mutant thereof having all the identifying characteristics thereof; 20% of a biologically pure culture of not less than about 1×10¹¹ CFU/g Bacillus subtilis CH201 deposited as DSM 17231, or a mutant thereof having all the identifying characteristics thereof; 10% maltodextrin; 40% calcium carbonate; and 10% silica. In embodiments, the composition applied to the seed improves plant growth and/or yield in the presence of a plant pathogenic nematode, improves plant resistance to a plant pathogenic nematode, or reduces plant infection by a plant pathogenic nematode, and combinations thereof.

In embodiments, a method for controlling plant pathogenic nematodes is provided, the method comprising: delivering to seed of a plant, roots of a plant, or soil surrounding a plant a liquid composition in the form of a suspension concentrate or an oil dispersion comprising by weight percent: 0.5-40% of a biologically pure culture of not less than about 1×10¹¹ CFU/g Bacillus licheniformis CH200 deposited as DSM 17236, or a mutant thereof having all the identifying characteristics thereof; 0.5-40% of a biologically pure culture of not less than about 1×10¹¹ CFU/g Bacillus subtilis CH201 deposited as DSM 17231, or a mutant thereof having all the identifying characteristics thereof; a liquid carrier; a surface active agent; and an adjuvant. In embodiments, the adjuvant is selected from the group consisting of preservatives, biocides or biostatic agents, thickeners, antifoams, and antifreezes, viscosity modifiers, biocides or biostatic agents, crystallization inhibitors, suspending agents, dyes, anti-oxidants, foaming agents, light absorbers, mixing auxiliaries, complexing agents, neutralizing or pH-modifying substances and buffers, corrosion inhibitors, fragrances, wetting agents, take-up enhancers, micronutrients, plasticizers, glidants, lubricants, and dispersants. The composition may be optionally further diluted with water. In embodiments, the composition when delivered to seed of the plant, roots of the plant, or soil surrounding the plant improves the plant growth and/or yield in the presence of a plant pathogenic nematode, improves the plant resistance to a plant pathogenic nematode, or reduces the plant infection by a plant pathogenic nematode, and combinations thereof.

In embodiments, the liquid composition is a suspension concentrate comprising water and at least one surface active agent, and one or more additional adjuvants selected from thickeners, solvents, preservatives, antifreeze agents, and antifoam agents.

In embodiments, the liquid composition is a suspension concentrate comprising from 1 to 10 weight % of the Bacillus licheniformis culture or a mutant thereof having all the identifying characteristics thereof; from 1 to 10 weight % of the Bacillus subtilis culture or a mutant thereof having all the identifying characteristics thereof; 1 to 5 weight % of one or more surface active agent; and at least one thickener, solvent, preservative, antifreeze agent, or antifoam agent each independently comprising up to about 1 weight % of the composition.

In embodiments of the method, the plant pathogenic nematode comprises a Meloidogyne spp. or a Pratylenchus. In embodiments of the method, the plant comprises soybean, cotton, dry bean, corn, sugarcane, coffee, or potato. In embodiments, delivery of the composition improves plant yield in the presence of a plant nematode pathogen.

In the articles of manufacture and in the methods for applying the composition to a plant seed, the plant seed can be a wide variety of plant seeds including, for example, seed of monocots, dicots, cereals, Corn, Sweet Corn, Popcorn, Seed Corn, Silage Corn, Field Corn, Rice, Wheat, Barley, Sorghum, Brassica Vegetables, Broccoli, Cabbage, Cauliflower, Brussels Sprouts, Collards, Kale, Mustard Greens, Kohlrabi, Bulb Vegetables, Onion, Garlic, Shallots, Fruiting Vegetables, Pepper, Tomato, Eggplant, Ground Cherry, Tomatillo, Okra, Grape, Herbs/Spices, Cucurbit Vegetables, Cucumber, Cantaloupe, Melon, Muskmelon, Squash, Watermelon, Pumpkin, Eggplant, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Cotton, Flax, Peanut, Rapeseed, Safflower, Sesame, Sunflower, Soybean, Root/Tuber and Corm Vegetables, Carrot, Potato, Sweet Potato, Beets, Ginger, Horseradish, Radish, Ginseng, Turnip, Sugarcane, Sugarbeet, Coffee, Grass, or Turf grass. In one embodiment, the plant seed can comprise soybean, dry bean, cotton, or corn. In embodiments, delivery of the composition to the plant seed improves plant yield in the presence of a plant nematode pathogen.

In the articles of manufacture and in the methods of the invention for applying the composition to a plant seed, the plant pathogenic nematode can include a Meloidogyne spp. or a Pratylenchus nematode.

In the articles of manufacture and in the methods for applying the composition to a plant seed, the plant seed can be further treated with one or a combination of a microbial, a biological, or a chemical insecticide, fungicide, nematicide, bacteriocide, herbicide, plant extract, plant growth regulator, or fertilizer present in an amount suitable to benefit plant growth and/or to confer protection against a pathogenic infection in the plant. The chemical fungicide can be one or a combination of mefenoxam, fluopyram, chlorothalonil, thiophanate-methyl, fludioxonil, metalaxyl, or sedaxane. The chemical insecticide can be one or a combination of imidacloprid, bifenthrin, thiamethoxam, pyrethroids, tefluthrin, zeta-cypermethrin, organophosphates, chlorethoxyphos, chlorpyrifos, tebupirimphos, cyfluthrin, fiproles, fipronil, nicotinoids, or clothianidin.

In embodiments, a method is provided for controlling plant pathogenic nematodes, the method comprising: delivering to seed of a plant, roots of a plant, or soil surrounding a plant a composition comprising by weight percent: 5-20% of a biologically pure culture of not less than about 1×10¹¹ CFU/g Bacillus licheniformis CH200 deposited as DSM 17236, or a mutant thereof having all the identifying characteristics thereof; 5-20% of a biologically pure culture of not less than about 1×10¹¹ CFU/g Bacillus subtilis CH201 deposited as DSM 17231, or a mutant thereof having all the identifying characteristics thereof; 5-15% maltodextrin; 35-45% calcium carbonate; and 5-15% silica. In embodiments, the composition delivered to seed of the plant, roots of the plant, or soil surrounding the plant improves the plant growth and/or yield in the presence of a plant pathogenic nematode, improves the plant resistance to a plant pathogenic nematode, or reduces the plant infection by a plant pathogenic nematode, and combinations thereof.

In one embodiment of the method for controlling plant pathogenic nematodes by delivering the composition of the invention to seed of a plant, roots of a plant, or soil surrounding a plant, the composition can comprise by weight percent: 20% of a biologically pure culture of not less than about 1×10¹¹ CFU/g Bacillus licheniformis CH200 deposited as DSM 17236, or a mutant thereof having all the identifying characteristics thereof; 20% of a biologically pure culture of not less than about 1×10¹¹ CFU/g Bacillus subtilis CH201 deposited as DSM 17231, or a mutant thereof having all the identifying characteristics thereof; 10% maltodextrin; 40% calcium carbonate; and 10% silica. In embodiments, the composition applied to the seed improves plant growth and/or yield in the presence of a plant pathogenic nematode, improves plant resistance to a plant pathogenic nematode, or reduces plant infection by a plant pathogenic nematode, and combinations thereof.

In embodiments of the method for controlling plant pathogenic nematodes by delivering a composition of the invention to seed of a plant, roots of a plant, or soil surrounding a plant, the plant pathogenic nematode can include a Meloidogyne spp. or a Pratylenchus nematode.

In embodiments of the method for controlling plant pathogenic nematodes by delivering a composition of the invention to seed of a plant, roots of a plant, or soil surrounding a plant, the plant can be a wide variety of plants including, for example, monocots, dicots, cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hardwood cuttings, Chestnuts, Oak, Maple, Coffee, Sugarcane, or Sugarbeet. In one embodiment, the plant can comprise soybean, cotton, dry bean, corn, sugarcane, coffee, or potato. In embodiments, delivery of the composition improves plant yield in the presence of a plant nematode pathogen.

In some embodiments, the method for controlling plant pathogenic nematodes by delivering the composition of the invention to seed of a plant, roots of a plant, or soil surrounding a plant can further include delivering to seed of the plant, roots of the plant, or soil surrounding the plant one or a combination of a microbial, a biological, or a chemical insecticide, fungicide, nematicide, bacteriocide, herbicide, plant extract, plant growth regulator, or fertilizer present in an amount suitable to benefit plant growth and/or to confer protection against a pathogenic infection in the plant. The chemical fungicide can be one or a combination of mefenoxam, fluopyram, chlorothalonil, thiophanate-methyl, fludioxonil, metalaxyl, or sedaxane. The chemical insecticide can be one or a combination of imidacloprid, thiamethoxam, pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, or clothianidin.

In embodiments, the chemical insecticide comprises chlorantraniliprole, cyantraniliprole, cyclaniliprole, or indoxacarb, optionally in combination with one or more of imidacloprid, bifenthrin, thiamethoxam, pyrethroids, tefluthrin, zeta-cypermethrin, organophosphates, chlorethoxyphos, chlorpyrifos, tebupirimphos, cyfluthrin, fiproles, fipronil, nicotinoids, or clothianidin.

In one embodiment, the chemical insecticide can include bifenthrin in a formulation compatible with a liquid fertilizer. The formulation compatible with a liquid fertilizer can include a hydrated aluminum-magnesium silicate and at least one dispersant. The hydrated aluminum-magnesium silicate can be selected from the group consisting of montmorillonite and attapulgite. The at least one dispersant can be selected from a sucrose ester, a lignosulfonate, an alkylpolyglycoside, a naphthalenesulfonic acid formaldehyde condensate and a phosphate ester. The phosphate ester can be selected from a nonyl phenol phosphate ester and a tridecyl alcohol ethoxylated phosphate potassium salt. The bifenthrin insecticide can be present at a concentration ranging from 0.1 g/ml to 0.2 g/ml. The bifenthrin insecticide can be present at a concentration of about 0.1715 g/ml. The term “in a formulation compatible with a liquid fertilizer” is intended to mean that the formulation is capable of dissolution or dispersion or emulsion in an aqueous solution to allow for mixing with a fertilizer for delivery to plants in a liquid formulation.

Suitable insecticides, herbicides, fungicides, and nematicides of the compositions and methods of the present invention can include the following:

Insecticides: AO) various insecticides, including agrigata, al-phosphide, amblyseius, aphelinus, aphidius, aphidoletes, artimisinin, autographa californica NPV, azocyclotin, Bacillus subtilis, Bacillus thuringiensis-spp. aizawai, Bacillus thuringiensis spp. kurstaki, Bacillus thuringiensis, Beauveria, Beauveria bassiana, betacyfluthrin, biologicals, bisultap, brofluthrinate, bromophos-e, bromopropylate, Bt-Corn-GM, Bt-Soya-GM, capsaicin, cartap, celastrus-extract, chlorantraniliprole, chlorbenzuron, chlorethoxyfos, chlorfluazuron, chlorpyrifos-e, cnidiadin, cryolite, cyanophos, cyantraniliprole, cyclaniliprole, cyhalothrin, cyhexatin, cypermethrin, dacnusa, DCIP, dichloropropene, dicofol, diglyphus, diglyphus+dacnusa, dimethacarb, dithioether, dodecyl-acetate, emamectin, encarsia, EPN, eretmocerus, ethylene-dibromide, eucalyptol, fatty-acids, fatty-acids/salts, fenazaquin, fenobucarb (BPMC), fenpyroximate, flubrocythrinate, flufenzine, formetanate, formothion, furathiocarb, gamma-cyhalothrin, garlic-juice, granulosis-virus, harmonia, heliothis armigera NPV, inactive bacterium, indol-3-ylbutyric acid, iodomethane, iron, isocarbofos, isofenphos, isofenphos-m, isoprocarb, isothioate, kaolin, lindane, liuyangmycin, matrine, mephosfolan, metaldehyde, metarhizium-anisopliae, methamidophos, metolcarb (MTMC), mineral-oil, mirex, m-isothiocyanate, monosultap, myrothecium verrucaria, naled, neochrysocharis formosa, nicotine, nicotinoids, oil, oleic-acid, omethoate, orius, oxymatrine, paecilomyces, paraffin-oil, parathion-e, pasteuria, petroleum-oil, pheromones, phosphorus-acid, photorhabdus, phoxim, phytoseiulus, pirimiphos-e, plant-oil, plutella xylostella GV, polyhedrosis-virus, polyphenol-extracts, potassium-oleate, profenofos, prosuler, prothiofos, pyraclofos, pyrethrins, pyridaphenthion, pyrimidifen, pyriproxifen, quillay-extract, quinomethionate, rape-oil, rotenone, saponin, saponozit, sodium-compounds, sodium-fluosilicate, starch, steinernema, streptomyces, sulfluramid, sulphur, tebupirimfos, tefluthrin, temephos, tetradifon, thiofanox, thiometon, transgenics (e.g., Cry3Bb1), triazamate, trichoderma, trichogramma, triflumuron, verticillium, vertrine, isomeric insecticides (e.g., kappa-bifenthrin, kappa-tefluthrin), dichoromezotiaz, broflanilide, pyraziflumid; A1) the class of carbamates, including aldicarb, alanycarb, benfuracarb, carbaryl, carbofuran, carbosulfan, methiocarb, methomyl, oxamyl, pirimicarb, propoxur and thiodicarb; A2) the class of organophosphates, including acephate, azinphos-ethyl, azinphos-methyl, chlorfenvinphos, chlorpyrifos, chlorpyrifos-methyl, demeton-S-methyl, diazinon, dichlorvos/DDVP, dicrotophos, dimethoate, disulfoton, ethion, fenitrothion, fenthion, isoxathion, malathion, methamidaphos, methidathion, mevinphos, monocrotophos, oxymethoate, oxydemeton-methyl, parathion, parathion-methyl, phenthoate, phorate, phosalone, phosmet, phosphamidon, pirimiphos-methyl, quinalphos, terbufos, tetrachlorvinphos, triazophos and trichlorfon; A3) the class of cyclodiene organochlorine compounds such as endosulfan; A4) the class of fiproles, including ethiprole, fipronil, pyrafluprole and pyriprole; A5) the class of neonicotinoids, including acetamiprid, clothianidin, dinotefuran, imidacloprid, nitenpyram, thiacloprid and thiamethoxam; A6) the class of spinosyns such as spinosad and spinetoram; A7) chloride channel activators from the class of mectins, including abamectin, emamectin benzoate, ivermectin, lepimectin and milbemectin; A8) juvenile hormone mimics such as hydroprene, kinoprene, methoprene, fenoxycarb and pyriproxyfen; A9) selective homopteran feeding blockers such as pymetrozine, flonicamid and pyrifluquinazon; A10) mite growth inhibitors such as clofentezine, hexythiazox and etoxazole; A11) inhibitors of mitochondrial ATP synthase such as diafenthiuron, fenbutatin oxide and propargite; uncouplers of oxidative phosphorylation such as chlorfenapyr; A12) nicotinic acetylcholine receptor channel blockers such as bensultap, cartap hydrochloride, thiocyclam and thiosultap sodium; A13) inhibitors of the chitin biosynthesis type 0 from the benzoylurea class, including bistrifluron, diflubenzuron, flufenoxuron, hexaflumuron, lufenuron, novaluron and teflubenzuron; A14) inhibitors of the chitin biosynthesis type 1 such as buprofezin; A15) moulting disruptors such as cyromazine; A16) ecdyson receptor agonists such as methoxyfenozide, tebufenozide, halofenozide and chromafenozide; A17) octopamin receptor agonists such as amitraz; A18) mitochondrial complex electron transport inhibitors pyridaben, tebufenpyrad, tolfenpyrad, flufenerim, cyenopyrafen, cyflumetofen, hydramethylnon, acequinocyl or fluacrypyrim; A19) voltage-dependent sodium channel blockers such as indoxacarb and metaflumizone; A20) inhibitors of the lipid synthesis such as spirodiclofen, spiromesifen and spirotetramat; A21) ryanodine receptor-modulators from the class of diamides, including flubendiamide, the phthalamide compounds (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, chlorantraniliprole, cyclaniliprole and cyantraniliprole; A22) compounds of unknown or uncertain mode of action such as azadirachtin, amidoflumet, bifenazate, fluensulfone, piperonyl butoxide, pyridalyl, sulfoxaflor; or A23) sodium channel modulators from the class of pyrethroids, including acrinathrin, allethrin, bifenthrin, cyfluthrin, lambda-cyhalothrin, cypermethrin, alpha-cypermethrin, beta-cypermethrin, zeta-cypermethrin, deltamethrin, esfenvalerate, etofenprox, fenpropathrin, fenvalerate, flucythrinate, tau-fluvalinate, permethrin, silafluofen and tralomethrin.

Fungicides: B0) benzovindiflupyr, antiperonosporic, ametoctradin, amisulbrom, copper salts (e.g., copper hydroxide, copper oxychloride, copper sulfate, copper persulfate), boscalid, thiflumazide, flutianil, furalaxyl, thiabendazole, benodanil, mepronil, isofetamid, fenfuram, bixafen, fluxapyroxad, penflufen, sedaxane, coumoxystrobin, enoxastrobin, flufenoxystrobin, pyraoxystrobin, pyrametostrobin, triclopyricarb, fenaminstrobin, metominostrobin, pyribencarb, meptyldinocap, fentin acetate, fentin chloride, fentin hydroxide, oxytetracycline, chlozolinate, chloroneb, tecnazene, etridiazole, iodocarb, prothiocarb, Bacillus subtilis syn., Bacillus amyloliquefaciens (e.g., strains QST 713, FZB24, MBI600, D747), extract from Melaleuca alternifolia, pyrisoxazole, oxpoconazole, etaconazole, fenpyrazamine, fenpicoxamide, mefentrifluconazole, naftifine, terbinafine, validamycin, pyrimorph, valifenalate, fthalide, probenazole, isotianil, laminarin, estract from Reynoutria sachalinensis, phosphorous acid and salts, teclofthalam, triazoxide, pyriofenone, organic oils, potassium bicarbonate, chlorothalonil, fluoroimide; B1) azoles, including bitertanol, bromuconazole, cyproconazole, difenoconazole, diniconazole, enilconazole, epoxiconazole, fluquinconazole, fenbuconazole, flusilazole, flutriafol, hexaconazole, imibenconazole, ipconazole, metconazole, myclobutanil, penconazole, propiconazole, prothioconazole, simeconazole, triadimefon, triadimenol, tebuconazole, tetraconazole, triticonazole, prochloraz, pefurazoate, imazalil, triflumizole, cyazofamid, benomyl, carbendazim, thiabendazole, fuberidazole, ethaboxam, etridiazole and hymexazole, azaconazole, diniconazole-M, oxpoconazol, paclobutrazol, uniconazol, 1-(4-chloro-phenyl)-2-([1,2,4]triazol-1-yl)-cycloheptanol and imazalilsulfphate; B2) strobilurins, including azoxystrobin, dimoxystrobin, enestroburin, fluoxastrobin, kresoxim-methyl, methominostrobin, orysastrobin, picoxystrobin, pyraclostrobin, trifloxystrobin, enestroburin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate and methyl 2-(ortho-(2,5-dimethylphenyloxymethylene)-phenyl)-3-methoxyacrylate, 2-(2-(6-(3-chloro-2-methyl-phenoxy)-5-fluoro-pyrimidin-4-yloxy)-phenyl)-2-methoxyimino-N-methyl-acetamide and 3-methoxy-2-(2-(N-(4-methoxy-phenyl)-cyclopropanecarboximidoylsulfanylmethyl)-phenyl)-acrylic acid methyl ester; B3) carboxamides, including carboxin, benalaxyl, benalaxyl-M, fenhexamid, flutolanil, furametpyr, mepronil, metalaxyl, mefenoxam, ofurace, oxadixyl, oxycarboxin, penthiopyrad, isopyrazam, thifluzamide, tiadinil, 3,4-dichloro-N-(2-cyanophenyl)isothiazole-5-carboxamide, dimethomorph, flumorph, flumetover, fluopicolide (picobenzamid), zoxamide, carpropamid, diclocymet, mandipropamid, N-(2-(4-[3-(4-chlorophenyl)prop-2-ynyloxy]-3-methoxyphenyl)ethyl)-2-methanesulfonyl-amino-3-methylbutyramide, N-(2-(4-[3-(4-chloro-phenyl)prop-2-ynyloxy]-3-methoxy-phenyl)ethyl)-2-ethanesulfonylamino-3-methylbutyramide, methyl 3-(4-chlorophenyl)-3-(2-isopropoxycarbonyl-amino-3-methyl-butyrylamino)propionate, N-(4′-bromobiphenyl-2-yl)-4-difluoromethyl-2-methylthiazole-5-carboxamide, N-(4′-trifluoromethyl-biphenyl-2-yl)-4-difluoromethyl-2-methylthiazole-5-carboxamide, N-(4′-chloro-3′-fluorobiphenyl-2-yl)-4-difluoromethyl-2-methyl-thiazole-5-carboxamide, N-(3′,4′-dichloro-4-fluorobiphenyl-2-yl)-3-difluoro-methyl-1-methyl-pyrazole-4-carboxamide, N-(3′,4′-dichloro-5-fluorobiphenyl-2-yl)-3-difluoromethyl-1-methylpyrazole-4-carboxamide, N-(2-cyano-phenyl)-3,4-dichloroisothiazole-5-carboxamide, 2-amino-4-methyl-thiazole-5-carboxanilide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxamide, N-(4′-chloro-3′,5-difluoro-biphenyl-2-yl)-3-difluoromethyl-1-methyl-1H-pyrazole-4-carboxamide, N-(4′-chloro-3′,5-difluoro-biphenyl-2-yl)-3-trifluoromethyl-1-methyl-1H-pyrazole-4-carboxamide, N-(3′,4′-dichloro-5-fluoro-biphenyl-2-yl)-3-trifluoromethyl-1-methyl-1H-pyrazole-4-carboxamide, N-(3′,5-difluoro-4′-methyl-biphenyl-2-yl)-3-difluoromethyl-1-methyl-1H-pyrazole-4-carboxamide, N-(3′,5-difluoro-4′-methyl-biphenyl-2-yl)-3-trifluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoro-methyl-1-methyl-1H-pyrazole-4-carboxamide, fluopyram, N-(3-ethyl-3,5-5-trimethyl-cyclohexyl)-3-formylamino-2-hydroxy-benzamide, oxytetracyclin, silthiofam, N-(6-methoxy-pyridin-3-yl) cyclopropanecarboxamide, 2-iodo-N-phenyl-benzamide, N-(2-bicyclo-propyl-2-yl-phenyl)-3-difluormethyl-1-methylpyrazol-4-ylcarboxamide, N-(3′,4′,5′-trifluorobiphenyl-2-yl)-1,3-dimethylpyrazol-4-ylcarboxamide, N-(3′,4′,5′-trifluorobiphenyl-2-yl)-1,3-dimethyl-5-fluoropyrazol-4-yl-carboxamide, N-(3′,4′,5′-trifluorobiphenyl-2-yl)-5-chloro-1,3-dimethyl-pyrazol-4-ylcarboxamide, N-(3′,4′,5′-trifluorobiphenyl-2-yl)-3-fluoromethyl-1-methylpyrazol-4-ylcarboxamide, N-(3′,4′,5′-trifluorobiphenyl-2-yl)-3-(chlorofluoromethyl)-1-methylpyrazol-4-ylcarboxamide, N-(3′,4′,5′-trifluorobiphenyl-2-yl)-3-difluoromethyl-1-methylpyrazol-4-ylcarboxamide, N-(3′,4′,5′-trifluorobiphenyl-2-yl)-3-difluoromethyl-5-fluoro-1-methylpyrazol-4-ylcarboxamide, N-(3′,4′,5′-trifluorobiphenyl-2-yl)-5-chloro-3-difluoromethyl-1-methylpyrazol-4-ylcarboxamide, N-(3′,4′,5′-trifluorobiphenyl-2-yl)-3-(chlorodifluoromethyl)-1-methylpyrazol-4-ylcarboxamide, N-(3′,4′,5′-trifluorobiphenyl-2-yl)-1-methyl-3-trifluoromethylpyrazol-4-ylcarboxamide, N-(3′,4′,5′-trifluorobiphenyl-2-yl)-5-fluoro-1-methyl-3-trifluoromethylpyrazol-4-ylcarboxamide, N-(3′,4′,5′-trifluorobiphenyl-2-yl)-5-chloro-1-methyl-3-trifluoromethylpyrazol-4-ylcarboxamide, N-(2′,4′,5′-trifluorobiphenyl-2-yl)-1,3-dimethylpyrazol-4-ylcarboxamide, N-(2′,4′,5′-trifluorobiphenyl-2-yl)-1,3-dimethyl-5-fluoropyrazol-4-ylcarboxamide, N-(2′,4′,5′-trifluorobiphenyl-2-yl)-5-chloro-1,3-dimethylpyrazol-4-ylcarboxamide, N-(2′,4′,5′-trifluorobiphenyl-2-yl)-3-fluoromethyl-1-methylpyrazol-4-ylcarboxamide, N-(2′,4′,5′-trifluorobiphenyl-2-yl)-3-(chlorofluoromethyl)-1-methylpyrazol-4-ylcarboxamide, N-(2′,4′,5′-trifluorobiphenyl-2-yl)-3-difluoromethyl-1-methylpyrazol-4-ylcarboxamide, N-(2′,4′,5′-trifluorobiphenyl-2-yl)-3-difluoromethyl-5-fluoro-1-methylpyrazol-4-ylcarboxamide, N-(2′,4′,5′-trifluorobiphenyl-2-yl)-5-chloro-3-difluoromethyl-1-methylpyrazol-4-ylcarboxamide, N-(2′,4′,5′-trifluorobiphenyl-2-yl)-3-(chlorodifluoromethyl)-1-methylpyrazol-4-ylcarboxamide, N-(2′,4′,5′-trifluorobiphenyl-2-yl)-1-methyl-3-trifluoromethylpyrazol-4-ylcarboxamide, N-(2′,4′,5′-trifluorobiphenyl-2-yl)-5-fluoro-1-methyl-3-trifluoromethylpyrazol-4-ylcarboxamide, N-(2′,4′,5′-trifluorobiphenyl-2-yl)-5-chloro-1-methyl-3-trifluoromethylpyrazol-4-ylcarboxamide, N-(3′,4′-dichloro-3-fluorobiphenyl-2-yl)-1-methyl-3-trifluoromethyl-1H-pyrazole-4-carboxamide, N-(3′,4′-dichloro-3-fluorobiphenyl-2-yl)-1-methyl-3-difluoromethyl-1H-pyrazole-4-carboxamide, N-(3′,4′-difluoro-3-fluorobiphenyl-2-yl)-1-methyl-3-trifluoromethyl-1H-pyrazole-4-carboxamide, N-(3′,4′-difluoro-3-fluorobiphenyl-2-yl)-1-methyl-S-difluoromethyl-1H-pyrazole-4-carboxamide, N-(3′-chloro-4′-fluoro-3-fluorobiphenyl-2-yl)-1-methyl-3-difluoromethyl-1H-pyrazole-4-carboxamide, N-(3′,4′-dichloro-4-fluorobiphenyl-2-yl)-1-methyl-3-trifluoromethyl-1H-pyrazole-4-carboxamide, N-(3′,4′-difluoro-4-fluorobiphenyl-2-yl)-1-methyl-S-trifluoromethyl-1H-pyrazole-4-carboxamide, N-(3′,4′-dichloro-4-fluorobiphenyl-2-yl)-1-methyl-3-difluoromethyl-1H-pyrazole-4-carboxamide, N-(3′,4′-difluoro-4-fluorobiphenyl-2-yl)-1-methyl-3-difluoromethyl-1H-pyrazole-4-carboxamide, N-(3′-chloro-4′-fluoro-4-fluorobiphenyl-2-yl)-1-methyl-5-difluoromethyl-1H-pyrazole-4-carboxamide, N-(3′,4′-dichloro-5-fluorobiphenyl-2-yl)-1-methyl-3-trifluoromethyl-1H-pyrazole-4-carboxamide, N-(3′,4′-difluoro-5-fluorobiphenyl-2-yl)-1-methyl-3-trifluoromethyl-1H-pyrazole-4-carboxamide, N-(3′,4′-dichloro-5-fluorobiphenyl-2-yl)-1-methyl-S-difluoromethyl-1H-pyrazole-carboxamide, N-(3′,4′-difluoro-5-fluorobiphenyl-2-yl)-1-methyl-3-difluoromethyl-1H-pyrazole-4-carboxamide, N-(3′,4′-dichloro-5-fluorobiphenyl-2-yl)-1,3-dimethyl-1H-pyrazole-4-carboxamide, N-(3′-chloro-4′-fluoro-5-fluorobiphenyl-2-yl)-1-methyl-3-difluoromethyl-1H-pyrazole-4-carboxamide, N-(4′-fluoro-4-fluorobiphenyl-2-yl)-1-methyl-3-trifluoromethyl-1H-pyrazole-4-carboxamide, N-(4′-fluoro-5-fluorobiphenyl-2-yl)-1-methyl-3-trifluoromethyl-1H-pyrazole-4-carboxamide, N-(4′-chloro-5-fluorobiphenyl-2-yl)-1-methyl-3-trifluoromethyl-1H-pyrazole-4-carboxamide, N-(4′-methyl-5-fluorobiphenyl-2-yl)-1-methyl-3-trifluoromethyl-1H-pyrazole-4-carboxamide, N-(4′-fluoro-5-fluorobiphenyl-2-yl)-1,3-dimethyl-1H-pyrazole-4-carboxamide, N-(4′-chloro-5-fluorobiphenyl-2-yl)-1,3-dimethyl-1H-pyrazole-4-carboxamide, N-(4′-methyl-5-fluorobiphenyl-2-yl)-1,3-dimethyl-1H-pyrazole-4-carboxamide, N-(4′-fluoro-6-fluorobiphenyl-2-yl)-1-methyl-3-trifluoromethyl-1H-pyrazole-4-carboxamide, N-(4′-chloro-6-fluorobiphenyl-2-yl)-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[4′-(trifluoromethylthio)-biphenyl-2-yl]-3-difluoromethyl-1-methyl-1H-pyrazole-4-carboxamide and N-[4′-(trifluoromethylthio)-biphenyl-2-yl]-1-methyl-3-trifluoromethyl-1-methyl-1H-pyrazole-4-carboxamide; B4) heterocyclic compounds, including fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, probenazole, 5-chloro-7-(4-methyl-piperidin-1-yl)-6-(2,4,6-trifluorophenyl)-[1,2,4]triazolo[1,5-a]pyrimidine, anilazine, diclomezine, pyroquilon, proquinazid, tricyclazole, 2-butoxy-6-iodo-3-propylchromen-4-one, acibenzolar-S-methyl, captafol, captan, dazomet, folpet, fenoxanil, quinoxyfen, N,N-dimethyl-3-(3-bromo-6-fluoro-2-methylindole-1-sulfonyl)-[1,2,4]triazole-1-sulfonamide, 5-ethyl-6-octyl-[1,2,4]triazolo[1,5-a]pyrimidin-2,7-diamine, 2,3,5,6-tetrachloro-4-methanesulfonyl-pyridine, 3,4,5-trichloro-pyridine-2,6-di-carbonitrile, N-(1-(5-bromo-3-chloro-pyridin-2-yl)-ethyl)-2,4-dichloro-nicotinamide, N-((5-bromo-3-chloropyridin-2-yl)-methyl)-2,4-dichloro-nicotinamide, diflumetorim, nitrapyrin, dodemorphacetate, fluoroimid, blasticidin-S, chinomethionat, debacarb, difenzoquat, difenzoquat-methylsulphat, oxolinic acid and piperalin; B5) carbamates, including mancozeb, maneb, metam, methasulphocarb, metiram, ferbam, propineb, thiram, zineb, ziram, diethofencarb, iprovalicarb, benthiavalicarb, propamocarb, propamocarb hydrochlorid, 4-fluorophenyl N-(1-(1-(4-cyanophenyl)-ethanesulfonyl)but-2-yl)carbamate, methyl 3-(4-chloro-phenyl)-3-(2-isopropoxycarbonylamino-3-methyl-butyrylamino)propanoate; or B6) other fungicides, including guanidine, dodine, dodine free base, iminoctadine, guazatine, antibiotics: kasugamycin, oxytetracyclin and its salts, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, tolclofos-methyl, organochlorine compounds: dichlofluanid, flusulfamide, hexachloro-benzene, phthalide, pencycuron, quintozene, thiophanate, thiophanate-methyl, tolylfluanid, others: cyflufenamid, cymoxanil, dimethirimol, ethirimol, furalaxyl, metrafenone and spiroxamine, guazatine-acetate, iminoc-tadine-triacetate, iminoctadine-tris(albesilate), kasugamycin hydrochloride hydrate, dichlorophen, pentachlorophenol and its salts, N-(4-chloro-2-nitro-phenyl)-N-ethyl-4-methyl-benzenesulfonamide, dicloran, nitrothal-isopropyl, tecnazen, biphenyl, bronopol, diphenylamine, mildiomycin, oxincopper, prohexadione calcium, N-(cyclopropylmethoxyimino-(6-difluoromethoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluormethyl-4-(3-trimethylsilanyl-propoxy)-phenyl)-N-ethyl-N-methylformamidine and N′-(5-difluormethyl-2-methyl-4-(3-trimethylsilanyl-propoxy)-phenyl)-N-ethyl-N-methyl formamidine.

Herbicides: C1) acetyl-CoA carboxylase inhibitors (ACC), for example cyclohexenone oxime ethers, such as alloxydim, clethodim, cloproxydim, cycloxydim, sethoxydim, tralkoxydim, butroxydim, clefoxydim or tepraloxydim; phenoxyphenoxypropionic esters, such as clodinafop-propargyl, cyhalofop-butyl, diclofop-methyl, fenoxaprop-ethyl, fenoxaprop-P-ethyl, fenthiapropethyl, fluazifop-butyl, fluazifop-P-butyl, haloxyfop-ethoxyethyl, haloxyfop-methyl, haloxyfop-P-methyl, isoxapyrifop, propaquizafop, quizalofop-ethyl, quizalofop-P-ethyl or quizalofop-tefuryl; or arylaminopropionic acids, such as flamprop-methyl or flamprop-isopropyl; C2 acetolactate synthase inhibitors (ALS), for example imidazolinones, such as imazapyr, imazaquin, imazamethabenz-methyl (imazame), imazamox, imazapic or imazethapyr; pyrimidyl ethers, such as pyrithiobac-acid, pyrithiobac-sodium, bispyribac-sodium. KIH-6127 or pyribenzoxym; sulfonamides, such as florasulam, flumetsulam or metosulam; or sulfonylureas, such as amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, halosulfuron-methyl, imazosulfuron, metsulfuron-methyl, nicosulfuron, primisulfuron-methyl, prosulfuron, pyrazosulfuron-ethyl, rimsulfuron, sulfometuron-methyl, thifensulfuron-methyl, triasulfuron, tribenuron-methyl, triflusulfuron-methyl, tritosulfuron, sulfosulfuron, foramsulfuron or iodosulfuron; C3) amides, for example allidochlor (CDAA), benzoylprop-ethyl, bromobutide, chiorthiamid, diphenamid, etobenzanidibenzchlomet), fluthiamide, fosamin or monalide; C4) auxin herbicides, for example pyridinecarboxylic acids, such as clopyralid or picloram; or 2,4-D or benazolin; C5) auxin transport inhibitors, for example naptalame or diflufenzopyr; C6) carotenoid biosynthesis inhibitors, for example benzofenap, clomazone (dimethazone), diflufenican, fluorochloridone, fluridone, pyrazolynate, pyrazoxyfen, isoxaflutole, isoxachlortole, mesotrione, sulcotrione (chlormesulone), ketospiradox, flurtamone, norflurazon or amitrol; C7) enolpyruvylshikimate-3-phosphate synthase inhibitors (EPSPS), for example glyphosate or sulfosate; C8) glutamine synthetase inhibitors, for example bilanafos (bialaphos) or glufosinate-ammonium; C9) lipid biosynthesis inhibitors, for example anilides, such as anilofos or mefenacet; chloroacetanilides, such as dimethenamid, S-dimethenamid, acetochlor, alachlor, butachlor, butenachlor, diethatyl-ethyl, dimethachlor, metazachlor, metolachlor, S-metolachlor, pretilachlor, propachlor, prynachlor, terbuchlor, thenylchlor or xylachlor; thioureas, such as butylate, cycloate, di-allate, dimepiperate, EPIC. esprocarb, molinate, pebulate, prosulfocarb, thiobencarb (benthiocarb), tri-allate or vemolate; or benfuresate or perfluidone; C10) mitosis inhibitors, for example carbamates, such as asulam, carbetamid, chlorpropham, orbencarb, pronamid (propyzamid), propham or tiocarbazil; dinitroanilines, such as benefin, butralin, dinitramin, ethalfluralin, fluchloralin, oryzalin, pendimethalin, prodiamine or trifluralin; pyridines, such as dithiopyr or thiazopyr; or butamifos, chlorthal-dimethyl (DCPA) or maleic hydrazide; C11) protoporphyrinogen IX oxidase inhibitors, for example diphenyl ethers, such as acifluorfen, acifluorfen-sodium, aclonifen, bifenox, chlomitrofen (CNP), ethoxyfen, fluorodifen, fluoroglycofen-ethyl, fomesafen, furyloxyfen, lactofen, nitrofen, nitrofluorfen or oxyfluorfen; oxadiazoles, such as oxadiargyl or oxadiazon; cyclic imides, such as azafenidin, butafenacil, carfentrazone-ethyl, cinidon-ethyl, flumiclorac-pentyl, flumioxazin, flumipropyn, flupropacil, fluthiacet-methyl, sulfentrazone or thidiazimin; or pyrazoles, such as ET-751.JV 485 or nipyraclofen; C12) photosynthesis inhibitors, for example propanil, pyridate or pyridafol; benzothiadiazinones, such as bentazone; dinitrophenols, for example bromofenoxim, dinoseb, dinoseb-acetate, dinoterb or DNOC; dipyridylenes, such as cyperquat-chloride, difenzoquat-methylsulfate, diquat or paraquat-dichloride; ureas, such as chlorbromuron, chlorotoluron, difenoxuron, dimefuron, diuron, ethidimuron, fenuron, fluometuron, isoproturon, isouron, linuron, methabenzthiazuron, methazole, metobenzuron, metoxuron, monolinuron, neburon, siduron or tebuthiuron; phenols, such as bromoxynil or ioxynil; chloridazon; triazines, such as ametryn, atrazine, cyanazine, desmein, dimethamethryn, hexazinone, prometon, prometryn, propazine, simazine, simetryn, terbumeton, terbutryn, terbutylazine or trietazine; triazinones, such as metamitron or metribuzin; uracils, such as bromacil, lenacil or terbacil; or biscarbamates, such as desmedipham or phenmedipham; C13) synergists, for example oxiranes, such as tridiphane; C14) CIS cell wall synthesis inhibitors, for example isoxaben or dichlobenil; C15) various other herbicides, for example dichloropropionic acids, such as dalapon; dihydrobenzofurans, such as ethofumesate; phenylacetic acids, such as chlorfenac (fenac); or aziprotryn, barban, bensulide, benzthiazuron, benzofluor, buminafos, buthidazole, buturon, cafenstrole, chlorbufam, chlorfenprop-methyl, chloroxuron, cinmethylin, cumyluron, cycluron, cyprazine, cyprazole, dibenzyluron, dipropetryn, dymron, eglinazin-ethyl, endothall, ethiozin, flucabazone, fluorbentranil, flupoxam, isocarbamid, isopropalin, karbutilate, mefluidide, monuron, napropamide, napropanilide, nitralin, oxaciclomefone, phenisopham, piperophos, procyazine, profluralin, pyributicarb, secbumeton, sulfallate (CDEC), terbucarb, triaziflam, triazofenamid or trimeturon; or their environmentally compatible salts.

Nematicides or bionematicides: Benomyl, cloethocarb, aldoxycarb, tirpate, diamidafos, fenamiphos, cadusafos, dichlofenthion, ethoprophos, fensulfothion, fosthiazate, heterophos, isamidofof, isazofos, phosphocarb, thionazin, imicyafos, mecarphon, acetoprole, benclothiaz, chloropicrin, dazomet, fluensulfone, 1,3-dichloropropene (telone), dimethyl disulfide, metam sodium, metam potassium, metam salt (all MITC generators), methyl bromide, biological soil amendments (e.g., mustard seeds, mustard seed extracts), steam fumigation of soil, allyl isothiocyanate (AITC), dimethyl sulfate, furfual (aldehyde).

Suitable plant growth regulators of the present invention include the following: Plant Growth Regulators: D1) Antiauxins, such as clofibric acid, 2,3,5-tri-iodobenzoic acid; D2) Auxins such as 4-CPA, 2,4-D, 2,4-DB, 2,4-DEP, dichlorprop, fenoprop, IAA, IBA, naphthaleneacetamide, α-naphthaleneacetic acids, 1-naphthol, naphthoxyacetic acids, potassium naphthenate, sodium naphthenate, 2,4,5-T; D3) cytokinins, such as 2iP, benzyladenine, 4-hydroxyphenethyl alcohol, kinetin, zeatin; D4) defoliants, such as calcium cyanamide, dimethipin, endothal, ethephon, merphos, metoxuron, pentachlorophenol, thidiazuron, tribufos; D5) ethylene inhibitors, such as aviglycine, 1-methylcyclopropene; D6) ethylene releasers, such as ACC, etacelasil, ethephon, glyoxime; D7) gametocides, such as fenridazon, maleic hydrazide; D8) gibberellins, such as gibberellins, gibberellic acid; D9) growth inhibitors, such as abscisic acid, ancymidol, butralin, carbaryl, chlorphonium, chlorpropham, dikegulac, flumetralin, fluoridamid, fosamine, glyphosine, isopyrimol, jasmonic acid, maleic hydrazide, mepiquat, piproctanyl, prohydrojasmon, propham, tiaojiean, 2,3,5-tri-iodobenzoic acid; D10) morphactins, such as chlorfluren, chlorflurenol, dichlorflurenol, flurenol; D11) growth retardants, such as chlormequat, daminozide, flurprimidol, mefluidide, paclobutrazol, tetcyclacis, uniconazole; D12) growth stimulators, such as brassinolide, brassinolide-ethyl, DCPTA, forchlorfenuron, hymexazol, prosuler, triacontanol; D13) unclassified plant growth regulators, such as bachmedesh, benzofluor, buminafos, carvone, choline chloride, ciobutide, clofencet, cyanamide, cyclanilide, cycloheximide, cyprosulfamide, epocholeone, ethychlozate, ethylene, fuphenthiourea, furalane, heptopargil, holosulf, inabenfide, karetazan, lead arsenate, methasulfocarb, prohexadione, pydanon, sintofen, triapenthenol, and trinexapac.

In embodiments, suitable insecticides may include ryanodine receptor-modulators, including, but not limited to, chlorantraniliprole, cyclaniliprole or cyantraniliprole, and/or voltage-dependent sodium channel blockers such as indoxacarb, optionally in combination with imidacloprid, bifenthrin, thiamethoxam, pyrethroids, tefluthrin, zeta-cypermethrin, organophosphates, chlorethoxyphos, chlorpyrifos, tebupirimphos, cyfluthrin, fiproles, fipronil, nicotinoids, and clothianidin.

In ceratin embodiments, suitable insecticides may include ryanodine receptor-modulators, including, but not limited to, chlorantraniliprole, cyclaniliprole, and cyantraniliprole.

In embodiments, suitable insecticides may include Bacillus subtilis, Bacillus thuringiensis-spp. aizawai, Bacillus thuringiensis spp. kurstaki, Bacillus thuringiensis, chlorantraniliprole, chlorethoxyfos, chlorpyrifos-e, cyantraniliprole, cyclaniliprole, cypermethrin, dichloropropene, flupyradifurone, gamma-cyhalothrin, profenofos, tebupirimfos, tefluthrin, tetraniliprole, kappa-bifenthrin, kappa-tefluthrin, carbofuran, carbosulfan, oxamyl, thiodicarb; chlorpyrifos, chlorpyrifos-methyl, diazinon, phorate, terbufos, fipronil, acetamiprid, clothianidin, imidacloprid, thiacloprid, thiamethoxam; abamectin, flonicamid, flubendiamide, bifenthrin, lambda-cyhalothrin, cyper-methrin, zeta-cypermethrin, and deltamethrin.

In certain embodiments, suitable insecticides may include clothianidin, thiamethoxam, imidacloprid, tefluthrin, fipronil, chlorpyrifos-e, bifenthrin, cypermethrin, tebupirimfos, zeta-cypermethrin, gamma-cyhalothrin, oxamyl, cadusafos, chlorantraniliprole, cyantraniliprole, cyclaniliprole, and tetraniliprole.

In embodiments, suitable fungicides may include antiperonosporic compounds, including, but not limited to: ametoctradin, amisulbrom, benthiavalicarb, cyazofamid, cymoxanil, dimethomorph, ethaboxam, famoxadone, fenamidone, flumetover, flumorph, fluopicolide, iprovalicarb, mandipropamid, valifenalate, benalaxyl, benalaxyl-M, furalaxyl, metalaxyl, and metalaxyl-M.

In embodiments, suitable fungicides may include thiabendazole, fluxapyroxad, penflufen, sedaxane, Bacillus subtilis syn., Bacillus amyloliquefaciens (e.g., strains QST 713, FZB24, MBI600, D747), bitertanol, cyproconazole, difenoconazole, fluquinconazole, flutriafol, ipconazole, myclobutanil, prothioconazole, triadimefon, triadimenol, tebuconazole, triticonazole, prochloraz, imazalil, benomyl, carbendazim, hymexazole, azoxystrobin, fluoxastrobin, pyraclostrobin, trifloxystrobin, carboxin, flutolanil, metalaxyl, mefenoxam, penthiopyrad, fluopyram, silthiofam, fluazinam, pyrimethanil, fludioxonil, iprodione, tricyclazole, captan, dazomet, mancozeb, metam, thiram, guazatine, tolclofos-methyl, pencycuron, thiophanate-methyl, fenpicoxamide, and mefentrifluconazole.

In certain embodiments, suitable fungicides may include fludioxonil, prothioconazole, mefenoxam, metalaxyl, tebuconazole, difenoconazole, thiram, carboxin, carbendazim, triticonazole, pencycuron, imazalil, pyraclostrobin, sedaxane, trifloxystrobin, fluquinconazole, fluoxastrobin, azoxystrobin, flutriafol, fluxapyroxad, penthiopyrad, fenpicoxamide, and mefentrifluconazole.

In embodiments, the fungicide can be one or a combination of fenpicoxamide, mefentrifluconazole, mefenoxam, fluopyram, chlorothalonil, thiophanate-methyl, fludioxonil, metalaxyl, or sedaxane.

In certain embodiments, the fungicide may be one or a combination of mefenoxam, fluopyram, chlorothalonil, thiophanate-methyl, fludioxonil, metalaxyl, or sedaxane.

In embodiments, suitable fungicides may include antiperonosporic compounds, including, but not limited to: ametoctradin, amisulbrom, benthiavalicarb, cyazofamid, cymoxanil, dimethomorph, ethaboxam, famoxadone, fenamidone, flumetover, flumorph, fluopicolide, iprovalicarb, mandipropamid, valifenalate, benalaxyl, benalaxyl-M, furalaxyl, metalaxyl, and metalaxyl-M.

In embodiments, suitable nematicides or bionematicides may include: benomyl, fenamiphos, cadusafos, ethoprophos, fosthiazate, chloropicrin, dazomet, fluensulfone, 1,3-dichloropropene (telone), metam sodium, metam potassium, metam salt (all MITC generators), methyl bromide, allyl isothiocyanate (AITC), fluazaindolizine (DPX-Q8U80), and tioxazafen.

In ceratin embodiments, suitable nematicides may include cadusafos, ethoprophos, fosthiazate, fluensulfone, oxamyl, fluazaindolizine (DPX-Q8U80), and tioxazafen.

The fertilizer can be a liquid fertilizer. The term “liquid fertilizer” refers to a fertilizer in a fluid or liquid form containing various ratios of nitrogen, phosphorous and potassium (for example, but not limited to, 10% nitrogen, 34% phosphorous and 0% potassium) and micronutrients, commonly known as starter fertilizers that are high in phosphorus and promote rapid and vigorous root growth.

Chemical formulations of the present invention can be in any appropriate conventional form, for example an emulsion concentrate (EC), a suspension concentrate (SC), a suspo-emulsion (SE), a capsule suspension (CS), a water dispersible granule (WG), an emulsifiable granule (EG), a water in oil emulsion (EO), an oil in water emulsion (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a dispersible concentrate (DC), a wettable powder (WP) or any technically feasible formulation in combination with agriculturally acceptable adjuvants.

In embodiments, the compositions can further contain one or more adjuvants and/or carriers. In at least one embodiment, the active ingredients comprising the bacillus species are present in total concentrations ranging between 0.01% to about 95 weight % of the agricultural composition, such as wherein each bacillus species is present in an amount independently selected from a lower limit of 1, 2, 3, 4 or 5 weight % to an upper limit of 10, 15, 20, 25, 40, 50, 60, 70, 80 or 90 weight % of the total composition. In another embodiment, agriculturally acceptable carriers constitute about 1% to about 98.5%, such as from a lower limit of 1, 2, 3, 4 or 5 weight % to an upper limit of 10, 15, 20, 25, 40, 50, 60, 70, 80 or 90 weight % of the total composition.

Adjuvants may include antimicrobials, preservative, biocide, surfactants, thickeners, antifoams, antifreezes, solvents, and co-solvents.

Surface active agents including surfactants, dispersants and emulsifiers, viscosity enhancing agents, and solvents and other adjuvants independently may constitute between about 0.1% to about 25% of the final formulation by weight. In embodiments, the compositions of this invention may be formulated as a suspension concentrate (SC), wettable powder (WP) or wettable granule (WG).

Liquid carriers include water, petroleum ether, vegetable oils, acid anhydrides, amyl acetate, butylene carbonate, cyclohexane, cyclohexanol, diacetone alcohol, 1,2-dichloropropane, diethanolamine, diethylene glycol, diethylene glycol abietate, diethylene glycol butyl ether, diethylene glycol ethyl ether, diethylene glycol methyl ether, 1,4-dioxane, dipropylene glycol, dipropylene glycol methyl ether, dipropylene glycol dibenzoate, diproxitol, alkylpyrrolidone, 2-ethylhexanol, ethylene carbonate, 1,1,1-trichloroethane, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isobornyl acetate, isooctane, isophorone, isopropyl myristate, lactic acid, laurylamine, mesityl oxide, methoxypropanol, methyl laurate, methyl octanoate, methyl oleate, methylene chloride, n-hexane, n-octylamine, octadecanoic acid, octylamine acetate, oleic acid, oleylamine, polyethylene glycol (PEG), propionic acid, propyl lactate, propylene carbonate, propylene glycol, propylene glycol methyl ether, triethyl phosphate, triethylene glycol, xylenesulfonic acid, paraffin, mineral oil, trichloroethylene, perchloroethylene, alcohols of higher molecular weight, such as amyl alcohol, tetrahydrofurfuryl alcohol, hexanol, octanol, liquid amides such as N,N-dimethyloctanamide, N,N-dimethyldecanamide, N-methyl-N-(2-propylheptyl)-acetamide, N-methyl-N-(2-propylheptyl)-formamide, N-methyl-2-pyrrolidone and the like. Preferably, liquid carriers are such that the biological active agents remain essentially unchanged in the composition until after it is applied to the locus of control. Water is generally the carrier of choice for diluting the concentrated formulations.

Suitable solid carriers include, for example, maltodextrin, talc, titanium dioxide, pyrophyllite clay, silica (silicon dioxide), attapulgite clay, kieselguhr, limestone, calcium carbonate, bentonite, calcium montmorillonite, cottonseed husks, wheat flour, soybean flour, pumice, wood flour, ground walnut shells, lignin and similar substances, and mixtures thereof. Notable solid carriers include maltodextrin, silica, calcium carbonate, and mixtures thereof.

In yet another aspect, the compositions of may contain: (i) Bacillus licheniformis as described herein; (ii) Bacillus subtilis as described herein; and (iii) at least one inactive component selected from the group consisting of at least one carrier, at least one antimicrobial used as a preservative or biocide, at least one surface active agent, at least one thickener, at least one antifoam, at least one antifreeze, at least one solvent, and at least one co-solvent.

Further components that may be used in such formulations include crystallization inhibitors, viscosity modifiers, suspending agents, dyes, anti-oxidants, foaming agents, light absorbers, mixing auxiliaries, antifoams, complexing agents, neutralizing or pH-modifying substances and buffers, corrosion inhibitors, fragrances, wetting agents, take-up enhancers, micronutrients, plasticizers, glidants, lubricants, dispersants, and also liquid and solid fertilizers.

The compositions may contain a surface-active substance from a very large variety of substances known in the art that are also commercially available. Surface-active substances (described herein generally as surfactants) may be anionic, cationic, non-ionic or polymeric and they can be used as surfactants, dispersants, emulsifiers, wetting agents or suspending agents or for other purposes.

Surfactants belong to different classes such as cationic surfactants, anionic surfactants, non-ionic surfactants, ionic surfactants, and amphoteric surfactants. According to the invention, the surfactant can be any surfactant or combination of two or more surfactants useful to disperse the biological active ingredients in the formulation or tank mix for application. The amounts of the surfactant in the compositions of this invention may range from about 1 to about 15%, or about 1 to about 10%, preferably about 3 to about 8%, and more preferably about 5 to about 7% w/w.

Examples of some preferred surfactants include cationic, non-ionic, anionic and/or amphoteric surfactants.

Non-ionic surfactants suitable for this invention include ethoxylated linear alcohols, ethoxylated alkyl phenol, alkyl EO/PO copolymer, polyalkylene glycol monobutyl ether ethoxylated fatty acids/oils, sorbitan laurate, polysorbate, sorbitan oleate, ethoxylated fatty acid alcohols, or alkyl phenols, alkanolamides or alkyloamides (such as diethanolamide, lauric acid monoisopropanolamide, and ethoxylated myristamide), xyethylene fatty acid esters, polyoxyethylene fatty alcohol ethers (such as alkylaryl polyglycol ethers), alkylphenol/alkylene oxide addition products, such as nonylphenol ethoxylate; alcohol/alkylene oxide addition products, such as tridecylalcohol ethoxylate.

Anionic surfactants include alkyl-, alkylaryl- and arylsulfonates or salts thereof (such as sodium, potassium or calcium salts of lauryl sarcosinate, alkylbenzenesulfonate, dodecylbenzenesulfonate, alkylnaphthalenesulfonates such as dibutylnaphthalenesulfonate, or C₁₄₋₁₆ olefin sulfonates), alkyl-, alkylaryl- and arylsulfates or salts thereof (such as sodium, potassium or calcium salts of tridedeth sulfate, lauryl sulfate, decyl sulfate, and diethanolammonium lauryl sulfate) protein hydrolysates, derivatives of polycarboxylic acid (such as ammonium lauryl ether carboxylate), olefin sulfonates (such as sodium alpha olefin sulfonate), sarcosinates (such as ammonium cyclohexyl palmitoyl taurinate), succinates (such as disodium N-octadecyl sulfosuccinamate), phosphorus derivatives (such as phosphoric acid esters and their equivalent salts).

Cationic surfactants include alkyl benzyltrimethylammonium chloride, ammonium lauryl sulfate and lauramine oxide.

Other surface active substances include soaps, such as sodium stearate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters.

Also suitable are silicone surfactants, especially polyalkyl-oxide-modified heptamethyltriloxanes which are commercially available e.g. as Silwet L-77®, and also perfluorinated surfactants.

Of these, some even more specific types of preferred surfactants include non-ionic linear or branched alcohol ethoxylate surfactants, anionic phosphoric acid ester surfactants (sometimes referred to as “phosphate ester” surfactants), and cationic ethoxylated tallow amine surfactants.

In another aspect, the composition may contain a thickener. Suitable thickeners are rice, starch, gum arabic, gum tragacanth, guar flour, British gum, starch ethers and starch esters, gum resins, galactomannans, magnesium aluminum silicate, xanthan gum, carrageenan, cellulose derivatives, methyl cellulose, carboxymethylcellulose, alginates and combinations thereof. Other known commercial products may include Lattice NTC 50, Lattice NTC 60, methocel, clay, and veegum silica.

In another embodiment, the compositions of this invention may contain an antifreeze agent such as glycerine, ethylene glycol, propylene glycol, urea, calcium chloride, sodium nitrate, magnesium chloride and ammonium sulfate.

Suitable preservatives include but are not limited to C₁₂ to C₁₅ alkyl benzoates, alkyl p-hydroxybenzoates, aloe vera extract, ascorbic acid, benzalkonium chloride, benzoic acid, benzoic acid esters of C₉ to C₁₅ alcohols, butylated hydroxytoluene, butylated hydroxyanisole, tert-butylhydroquinone, castor oil, cetyl alcohols, chlorocresol, citric acid, cocoa butter, coconut oil, diazolidinyl urea, diisopropyl adipate, dimethyl polysiloxane, DMDM hydantoin, ethanol, ethylenediaminetetraacetic acid, fatty acids, fatty alcohols, hexadecyl alcohol, hydroxybenzoate esters, iodopropynyl butylcarbamate, isononyl iso-nonanoate, jojoba oil, lanolin oil, mineral oil, oleic acid, olive oil, parabens, polyethers, polyoxypropylene butyl ether, polyoxypropylene cetyl ether, potassium sorbate, propyl gallate, silicone oils, sodium propionate, sodium benzoate, sodium bisulfite, sorbic acid, stearic fatty acid, sulfur dioxide, vitamin E, vitamin E acetate and derivatives, esters, salts and mixtures thereof. Preferred preservatives include sodium o-phenylphenate, 5-chloro-2-methyl-4-isothiazolin-3-one, 2-methyl-4-isothiazolin-3-one, and 1,2-benisothiazolin-3-one.

Antifoam agents such as Xiameter AFE-100, Dow Corning AFs, Dow Corning 1520, 1530, or 1540 may also be used in the presently claimed formulations.

In embodiments, a process is provided for preparing a composition described herein, the process comprising mixing, in a dry or powder form: a biologically pure culture of not less than about 1×10¹¹ CFU/g Bacillus licheniformis CH200 deposited as DSM 17236, or a mutant thereof having all the identifying characteristics thereof; a biologically pure culture of not less than about 1×10¹¹ CFU/g Bacillus subtilis CH201 deposited as DSM 17231, or a mutant thereof having all the identifying characteristics thereof; maltodextrin; calcium carbonate; and silica, wherein the mixing is in a ratio to effect the weight percent of the composition.

In another aspect, the compositions may be prepared by a process following the steps of combining the biological active ingredients in effective amounts with carriers and adjuvants as described herein. The formulated compositions can be prepared e.g. by mixing the biological active agents with the formulation components in order to obtain compositions in the form of finely divided solids, granules or dispersions. The active ingredients can also be formulated with other components, such as finely divided solids, mineral oils, oils of vegetable or animal origin, modified oils of vegetable or animal origin, organic solvents, water, surface-active substances or combinations thereof.

In another embodiment, the mixture may further undergo a milling process until suitable particle sizes ranging from about 1 to about 250 microns are obtained. The composition may have particle sizes of less than 250, less than 100 or preferably less than 50 microns. In a preferred embodiment, the mixture is milled until 90% of the particle size (D90) is less than about 50 microns.

One embodiment is directed to a composition comprising: the biological active ingredients; and iii) at least one formulation component selected from the group consisting of adjuvants for an SC formulation; adjuvants for a WP formulation; and adjuvants for a WG formulation.

In another embodiment, the composition is in the form of an SC comprising at least one surfactant, and one or more additional adjuvants selected from thickeners, solvents, preservatives, antifreeze agents, thickeners, and antifoam agents and water.

In an embodiment, SC comprises from 1 to 10 weight % of Bacillus licheniformis as described herein; from 1 to 10 weight % of Bacillus subtilis as described herein; 1 to 5 weight % of one or more surfactants; and optionally at least one thickener, solvent, preservative, antifreeze agent, or antifoam agent; and water. The optional thickener, solvent, preservative, antifreeze agent, or antifoam agent may each independently comprise up to about 1 weight % of the SC formulation. The SC comprises water in a complementary amount to all the other components to bring the total composition to 100 weight % (qs).

The composition may be in solid form, for example a WP or WG formulation. These formulations comprise at least one solid carrier as described above. In embodiments, WP or WG formulations may comprise from about 1 to about 30 weight %, such as from 1 to 10, 5 to 10, or 5 to 30 weight %, of Bacillus licheniformis as described herein; from about 1 to about 30 weight %, such as from 1 to 10, 5 to 10, or 5 to 30 weight %, of Bacillus subtilis as described herein; at least one solid carrier selected from the group consisting of maltodextrin, calcium carbonate and silica. In an embodiment, the composition may comprise from 5 to 10 weight % of Bacillus licheniformis as described herein; from 5 to 10 weight % of Bacillus subtilis as described herein; from about 80 to about 90 weight % of maltodextrin, and about 0.5 to about 2 weight % of silica.

In another embodiment, the composition comprises a wettable powder formulation comprising by weight %:

5-30% (such as 20%) of a biologically pure culture of not less than about 1×10¹⁰ CFU/g Bacillus lichenformis CH200 deposited as DSM 17236, or a mutant thereof having all the identifying characteristics thereof;

5-30% (such as 20%) of a biologically pure culture of not less than about 1×10¹⁰ CFU/g Bacillus subtilis CH201 deposited as DSM 17231, or a mutant thereof having all the identifying characteristics thereof;

5-15% (such as 10%) maltodextrin;

35-45% (such as 40%) calcium carbonate; and

5-15% (such as 10%) silica.

The composition may be useful in either plant seed treatment or in-furrow applications. For seed treatment, a solution of the composition can be applied to seed using standard seed treatment procedures. The composition may applied to untreated seeds or seeds that have been treated with at least one additional crop protection agent as described below. Alternatively, the composition may also be mixed with an additional crop protection agent for seed treatment or in-furrow applications.

In embodiments, the formulated compositions can be in the form of concentrates which are diluted prior to use, although ready-to-use formulations can also be made. Whereas commercial products will preferably be formulated as concentrates, the end user will normally employ dilute formulations for application to the soil or plant. The dilutions can be made, for example, with water, liquid fertilizers, micronutrients, biological organisms, oil or solvents.

In embodiments, the formulated compositions may additionally include an additive comprising an oil of vegetable or animal origin, a mineral oil, alkyl esters of such oils or mixtures of such oils and oil derivatives. The amount of oil additive in the composition according to the invention is generally from 0.01 to 10%, based on the spray mixture. For example, the oil additive can be added to the spray tank in the desired concentration after the spray mixture has been prepared. In embodiments, oil additives may comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow.

Where features or aspects are described in terms of a Markush group or other grouping of alternatives, those skilled in the art will recognized that the invention is also thereby described in terms of any individual member or subgroup of members of the Markush group or other group.

### EXAMPLES

The following EXAMPLES have been included to provide guidance to one of ordinary skill in the art for practicing representative embodiments of the presently disclosed subject matter. In light of the present disclosure and the general level of skill in the art, those of skill can appreciate that the following EXAMPLES are intended to be exemplary only and that numerous changes, modifications, and alterations can be employed without departing from the scope of the presently disclosed subject matter.

### Example 1

### Development of a Wettable Powder Formulation Containing Active Ingredients Bacillus licheniformis CH200 and Bacillus subtilis CH201 for Seed Treatment and/or In-Furrow Application

A wettable powder formulation was developed containing active ingredients Bacillus licheniformis CH200 and Bacillus subtilis CH201 to control plant disease and improve plant growth and yield. The formulation that was developed provides for both optimal seed adhesion and flowability properties, and also provides for solubility when applied in-furrow. The key was to achieve the seed adhesion properties without compromising solubility for the in-furrow application. An additional benefit of the formulation is that it provides enhanced shelf-life for the microorganisms by absorbing residual water. The formulation was developed to be flexible to allow for different modes of application in the field. As a WS (Wettable for Seed Treatment) or WP (Wetable Powder) the biological is a robust formulation for in-furrow and seed treatment applications.

The challenge was to find a combination of ingredients to provide a formulation that was stable physically, preserved the shelf life of the microorganisms, while also providing seed adhesion and flowability properties without compromising solubility/wettablility when applied in-furrow. In addition to the active ingredient microorganisms Bacillus licheniformis CH200 and Bacillus subtilis CH201, the following ingredients: calcium carbonate, maltodextrin, and silica were investigated to determine a concentration to provide seed adhesion and flowability properties without compromising solubility/wettablility when applied in-furrow and to provide optimal disease control and plant growth and yield promoting properties. A biologically pure culture at a concentration of not less than about 3×10¹¹ CFU/g of each of Bacillus licheniformis CH200 and Bacillus subtilis CH201 was used in the formulation studies to provide a final concentration in the formulations tested of not less than about 1×10¹¹ CFU/g of each of the CH200 and CH201 strains. The use of calcium carbonate promotes a better shelf-life for the microorganism by absorbing residual water. The use of maltdextrine was investigated to find a balance to achieve a biological formulation with good seed adhesion properties and also good solubility for in-furrow application.

The amount of maltodextrin was chosen based on the studies of concentration versus soybean seed adhesion as shown below in FIG. 1 and FIG. 2. The percentage of adhesion was determined based on what remained on the seed without consideration of what remained in the jar. Based on the results, the preferred amount of maltodextrin was determined to be 5-15%. Various formulations were produced for seed treatment testing by homogenizing the listed ingredients in powder form. It was discovered that simple mixing of the ingredients by homogenization was preferable to any form of grinding. Three formulations are shown below (Formulations T1, T2, and T3).

Formulation T1:

| | | | Concentration | Concentration |
|---|---|---|---|---|
| Raw-Materials | CAS Number | Function | (% p/p) | (g/Kg) |
| Bacillus licheniformis | — | Active | 20.00 | 200.00⁽¹⁾ |
| CH200 | | Ingredient | | |
| Bacillus subtilis CH201 | — | Active | 20.00 | 200.00⁽²⁾ |
| | | Ingredient | | |
| Maltodextrin | 9050-36-6 | Vehicle | 20.00 | 200.00 |
| Silica | 112945-52-5 | Vehicle | 10.00 | 100.00 |
| Calcium carbonate | 1317-65-3 | Vehicle | 30.00 | 300.00 |

| | | |
|---|---|---|
| Total | 100.00 | 1000.00 |

Formulation T2:

| | | | Concentration | Concentration |
|---|---|---|---|---|
| Raw-Materials | CAS Number | Function | (% p/p) | (g/Kg) |
| Bacillus licheniformis | — | Active | 20.00 | 200.00⁽¹⁾ |
| CH200 | | Ingredient | | |
| Bacillus subtilis CH201 | — | Active | 20.00 | 200.00⁽²⁾ |
| | | Ingredient | | |
| Maltodextrin | 9050-36-6 | Vehicle | 10.00 | 100.00 |
| Silica | 112945-52-5 | Vehicle | 10.00 | 100.00 |
| Calcium carbonate | 1317-65-3 | Vehicle | 40.00 | 400.00 |

| | | |
|---|---|---|
| Total | 100.00 | 1000.00 |

Formulation T3:

| | | | Concentration | Concentration |
|---|---|---|---|---|
| Raw-Materials | CAS Number | Function | (% p/p) | (g/Kg) |
| Bacillus licheniformis | — | Active | 20.00 | 200.00⁽¹⁾ |
| CH200 | | Ingredient | | |
| Bacillus subtilis CH201 | — | Active | 20.00 | 200.00⁽²⁾ |
| | | Ingredient | | |
| Silica | 112945-52-5 | Vehicle | 10.00 | 100.00 |
| Calcium carbonate | 1317-65-3 | Vehicle | 50.00 | 500.00 |

| | | |
|---|---|---|
| Total | 100.00 | 1000.00 |

For the seed treatment testing with these formulations, 2.4 mL of a stock solution of each formulation was mixed with 400 g soybean seed using standard seed treatment procedure. In addition, the seed treatment testing included use of two commercially available chemical fungicide products which are standard in treatment of soybean seed. The results of the seed treatment testing are shown in FIG. 3. It is clear from the test results that Formulation T2 (FIGS. 3C & 3D) has the superior seed treatment properties. In the production of Formulation T2, the order of addition of the ingredients was: 1) maltodextrin; 2) calcium carbonate; 3) silicon dioxide; 4) CH200; and 5) CH201 and the total homogenization time was 5 min.

Additional wettable powder formulations were prepared similarly.

Formulation T4:

| | Technical | Formulation |
|---|---|---|
| Ingredients | cfu/g | Nominal weight % |
| CH200, Bacillus Licheniformis | 8.60 × 10¹¹ | 20 |
| CH201, Bacillus Subtilis | 8.10 × 10¹¹ | 20 |
| maltodextrin | | 40 |
| Calcium carbonate | | 16 |
| silica | | 4 |

Formulation T5:

| | Technical | Formulation |
|---|---|---|
| Ingredients | cfu/g | Nominal weight % |
| CH200, Bacillus Licheniformis | 8.60 × 10¹¹ | 6.5 |
| CH201, Bacillus Subtilis | 8.10 × 10¹¹ | 6.5 |
| maltodextrin | | 86 |
| silica | | 1 |

A suspension concentrate was also prepared as Formulation SC1. The ingredients summarized below were mixed as described below to prepare a suspension concentrate.

| | Technical | Formulation | | cfu/g, ml |
|---|---|---|---|---|
| Ingredients | cfu/g | Weight (g) | Nominal weight % | cfu/g |
| CH200, Bacillus Licheniformis | 8.60 × 10¹¹ | 29.5 | 2.95 | 2.53 × 10¹⁰ |
| CH201, Bacillus Subtilis | 8.10 × 10¹¹ | 31.3 | 3.13 | 2.53 × 10¹⁰ |
| Potassium sorbate | | 1.0 | 0.10 | |
| Ascorbic acid | | 4.0 | 0.40 | |
| Modified sodium lignosulfate | | 15.0 | 1.50 | |
| Sodium tripolyphosphate | | 11.0 | 1.10 | |
| Urea | | 18.0 | 1.80 | |
| Sodium bisulfate | | 24.0 | 2.40 | |
| Glycerin | | 350.0 | 35.00 | |
| Dow AF (xiameter) antifoam | | 0.5 | 0.05 | |
| Citric acid | | 2.3 | 0.23 | |
| Na₃Citrate | | 0.1 | 0.01 | |
| Xanthan gum | | 0.2 | 0.02 | |
| Water | | 513.2 | 51.32 | |
| Total | | 1000 | 100 | |

A vessel equipped with an agitator was charged with the calculated amount of water to provide 1 kg of total formulation. Potassium sorbate, modified sodium lignosulfate and sodium tripolyphosphate were added to the vessel, and the mixture was agitated until all the salts were dissolved. Urea, ascorbic acid and sodium bisulfate were added, and the mixture was agitated until all ingredients were dissolved. The antifoam (xiameter) was added, followed by addition of FMCH001 and FMCH002 under vigorous agitation. The slurry was further homogenized until a particle size d90 of less than 20 urn, preferably less than 10 urn, was achieved. The pH of the slurry was adjusted with sodium citrate and citric acid, until a pH=4.0. Finally, glycerin and xanthan gum were added under agitation to produce the formulation as a slurry.

Additional SC formulations were prepared similarly, as described below.

| SC2 | Technical | Formulation | | cfu/g, ml |
|---|---|---|---|---|
| Ingredients | cfu/g | Weight (g) | Nominal weight % | cfu/g |
| CH200, Bacillus Licheniformis | 4.60 × 10¹¹ | 15.1 | 5.09 | 2.34 × 10¹⁰ |
| CH201, Bacillus Subtilis | 4.90 × 10¹¹ | 14.20 | 4.79 | 2.35 × 10¹⁰ |
| Potassium sorbate | | 1.0 | 0.3 | |
| Ascorbic acid | | 1.0 | 0.3 | |
| Sodium lignosulfate | | 4.0 | 1.3 | |
| Sodium polytriphosphate | | 10.0 | 3.4 | |
| Urea | | 2.0 | 0.7 | |
| Citric acid | | 5.0 | 1.7 | |
| Sucrose | | 120.0 | 40.5 | |
| Dow AF (xiameter) antifoam | | 0.3 | 0.1 | |
| Water | | 124.0 | 41.8 | |
| Total | | 296.6 | 100 | |

| SC3 | Technical | Formulation | | cfu/g, ml |
|---|---|---|---|---|
| Ingredients | cfu/g | Weight (g) | Nominal weight % | cfu/g |
| CH200, Bacillus Licheniformis | 8.2 × 10¹¹ | 10.0 | 3.65 | 2.99 × 10¹⁰ |
| CH201, Bacillus Subtilis | 4.70 × 10¹¹ | 17.4 | 6.35 | 2.99 × 10¹⁰ |
| Potassium phosphate | | 5.0 | 1.8 | |
| Potassium sorbate | | 1.0 | 0.4 | |
| Ascorbic acid | | 0.5 | 0.2 | |
| Sodium lignosulfate | | 5.0 | 1.7 | |
| Potassium phosphate | | 5.0 | 1.8 | |
| Sodium hexametaphosphate | | 25.0 | 9.1 | |
| Urea | | 50.0 | 18.2 | |
| Yeast extract | | 5.0 | 1.8 | |
| Glycerin | | 55.0 | 20.1 | |
| Dow AF (xiameter) antifoam | | 0.1 | 0.04 | |
| Water | | 100 | 36.5 | |
| Total | | 274.0 | 100 | |

| SC4 | Technical | Formulation | | cfu/g, ml |
|---|---|---|---|---|
| Ingredients | cfu/g | Weight (g) | Nominal weight % | cfu/g |
| CH200, Bacillus Licheniformis | 8.2 × 10¹¹ | 10.0 | 3.65 | 3.00 × 10¹⁰ |
| CH201, Bacillus Subtilis | 4.70 × 10¹¹ | 17.48 | 6.38 | 3.00 × 10¹⁰ |
| Potassium sorbate | | 1.0 | 0.4 | |
| Ascorbic acid | | 0.5 | 0.2 | |
| Sodium lignosulfate | | 5.0 | 1.9 | |
| Potassium phosphate | | 5.0 | 1.8 | |
| Sodium hexametaphosphate | | 25.0 | 9.1 | |
| Urea | | 50.0 | 18.2 | |
| Fertilizer | | 10.0 | 3.6 | |
| Glycerin | | 55.0 | 20.1 | |
| Dow AF (xiameter) antifoam | | 0.1 | 0.04 | |
| Water | | 95 | 34.7 | |
| Total | | 274.1 | 100 | |

| SC5 | Technical | Formulation | | cfu/g, ml |
|---|---|---|---|---|
| Ingredients | cfu/g | Weight (g) | Nominal weight % | cfu/g |
| CH200, Bacillus Licheniformis | 8.2 × 10¹¹ | 9.2 | 3.66 | 3.00 × 10¹⁰ |
| CH201, Bacillus Subtilis | 4.70 × 10¹¹ | 16.1 | 6.4 | 3.00 × 10¹⁰ |
| Potassium sorbate | | 1.0 | 0.4 | |
| Ascorbic acid | | 1.0 | 0.4 | |
| Sodium lignosulfate | | 4.2 | 1.7 | |
| Ethylene Oxide/Propylene Oxide | | 1.0 | 0.4 | |
| Block Copolymer | | | | |
| Citric acid-H₂0 | | 2.0 | 0.8 | |
| Sodium citrate | | 2.0 | 0.8 | |
| Attapulgite | | 4.75 | 1.9% | |
| Yeast extract | | 3.00 | 1.2% | |
| Glycerin | | 34.00 | 13.5% | |
| Water | | 68.0 | 27.0 | |
| Total | | 251.6 | 100 | |

| SC6 | Technical | Formulation | | cfu/g, ml |
|---|---|---|---|---|
| Ingredients | cfu/g | Weight (g) | Nominal weight % | cfu/g |
| CH200, Bacillus Licheniformis | 6.00 × 10¹¹ | 97.49 | 4.35 | 2.61 × 10¹⁰ |
| CH201, Bacillus Subtilis | 3.00 × 10¹¹ | 219.24 | 9.78 | 2.93 × 10¹⁰ |
| Potassium sorbate | | 7.5 | 0.3 | |
| Ascorbic acid | | 7.5 | 0.33 | |
| Sodium lignosulfate | | 30 | 1.3 | |
| Sodium polytriphosphate | | 22.5 | 1.0 | |
| Sodium bisulfate | | 66.0 | 2.9 | |
| Urea | | 15.0 | 0.7% | |
| Dow AF (xiameter) | | 2.25 | 0.1% | |
| Glycerin | | 675.00 | 30.1% | |
| Water | | 1100.0 | 49.1 | |
| Total | | 2242.5 | 100 | |

A Wettable Powder formulation was prepared by mixing the components described below.

| WP1 | Technical | Formulation | | cfu/g, ml |
|---|---|---|---|---|
| Ingredients | cfu/g | Weight (g) | Nominal weight % | cfu/g |
| CH200, Bacillus Licheniformis | 8.2 × 10¹¹ | 4.6 | 3.67 | 3.01 × 10¹⁰ |
| CH201, Bacillus Subtilis | 4.7 × 10¹¹ | 8.0 | 6.39 | 3.00 × 10¹⁰ |
| Yeast extract | | 10.0 | 8.0 | |
| Silica | | 5.0 | 4.0 | |
| Sodium lignosulfate | | 9 | 7.2 | |
| Monopotassium phosphate | | 12.0 | 9.6 | |
| Sodium hexametaphosphate | | 66.0 | 2.9 | |
| Urea | | 14.8 | 11.8 | |
| Total | | 129.4 | 100 | |

### Example 2

### Seed Treatment with Formulation of Bacillus Licheniformis CH200 and Bacillus Subtilis CH201 Benefits Growth and Increases Yield Under Nematode Pressure in Soybean, Cotton, Drybean and Corn

The wettable powder composition shown below in Table 1 is the T2 formulation described in EXAMPLE 1 and was used as a seed treatment in field trials on soybean, cotton, corn, and dry bean as described in the following studies. The wettable powder composition is herein referred to as “T2”. The T2 composition was prepared as described in EXAMPLE 1, and the final concentration of each of the CH200 and CH201 strains in the T2 composition is not less than about 1×10¹¹ CFU/g.

TABLE 1
Composition in powder form for seed treatment or
in-furrow application referred to herein as “T2.”

| Ingredients | W/W % |
|---|---|
| Bacillus licheniformis CH200 | 20 |
| Bacillus subtilis CH201 | 20 |
| Maltodextrin | 10 |
| Calcium carbonate | 40 |
| Silica | 10 |

In the field trials, all soybean, corn, and dry bean seeds were treated with MAXIM® XL (a.i. Fludioxonil 25 g/lt and a.i. Metalaxyl-M 10 g/lt; SYNGENTA, INC) at 0.2 L, which is referred to herein as “Check”, and all cotton seeds were treated with DEROSAL PLUS (a.i. Carbendazim; BAYER CROPSCIENCE, INC) at 0.6 L, which is referred to herein as “Check”. In addition, the T2 composition and commercially available products QUALITY (a.i. Thrichoderma; Laboratórios Biocontrole Farroupilha) and nematicide AVICTA® (a.i. Abamectin; SYNGENTA, INC) were applied to soy, cotton, and corn crops according to Table 2 below. In the case of dry bean, the commercial nematicide FURADAN (a.i. Carbofuran; FMC Corporation) was used instead of AVICTA®. The field trials were performed in Brazil (2-6 separate trials were performed for each of the crops) under natural disease pressure conditions that included low to high nematode pressure.

TABLE 2
Field trials protocol

| | | | | | | | High Load Formulation | | | |
|---|---|---|---|---|---|---|---|---|---|---|
| | | | | | | | CFU/100 kg | | | |
| | g or mL F.P./100 kg of seeds | | | | | | of seeds | | CFU/hectare | |
| | T2 | T2 | T2 | T2 | Quality | Avicta | Min | Max | Min | Max |
| Soybean | 20 | 40 | 70 | 100 | 50 | 70 | 5 × 10¹² | 2.5 × 10¹³ | 2.5 × 10¹² | 1.2 × 10¹³ |
| Cotton | 80 | 160 | 200 | 300 | 200 | 300 | 2 × 10¹³ | 7.5 × 10¹³ | 4 × 10¹² | 1.5 × 10¹³ |
| Drybeans | 40 | 80 | 100 | 120 | 100 | X | 1 × 10¹³ | 3 × 10¹³ | 8 x 10¹² | 2.4 × 10¹³ |
| Corn | 40 | 80 | 100 | 120 | 100 | 70 | 1 × 10¹³ | 3 × 10¹³ | 2 × 10¹² | 6 × 10¹² |

### Soybean

Six separate soybean field trials were performed as described above under a range of low, medium, and high nematode pressure. The data are shown below in Table 3, which shows the results of the average of the 6 trials in Brazil on standability, seedling height, root length, and yield.

TABLE 3
Soybean field trials (average of 6 trials in Brazil) of seed treatment
with the T2 formulation of B. Licheniformis CH200 and B. Subtilis
CH201 as compared to commercial products QUALITY
(a.i. Thrichoderma) and nematicide AVICTA (a.i. abamectin).*

| | | Seedling | | |
|---|---|---|---|---|
| | Standability | Height | Root length | |
| Seed Treatment | (plants/meter) | (cm) | (cm) | Yield |
| (g/100 kg) | Final | 35 DAE | 35 DAE | kg/hectare |
| Check | 12.6 | 31.2 | 17.4 | 3254 |
| T2 (20) | 12.6 | 32.1 | 19.9 | 3538 |
| T2 (40) | 12.7 | 33.7 | 19.6 | 3640 |
| T2 (70) | 12.9 | 33.9 | 20.0 | 3514 |
| T2 (100) | 13.0 | 34.3 | 20.0 | 3745 |
| QUALITY (50) | 12.1 | 34.2 | 19.2 | 3495 |
| AVICTA ® (70) | 12.3 | 33.1 | 19.5 | 3623 |

The results in Table 3 show that treating soybean seed with the T2 formulation of Bacillus licheniformis CH200 and Bacillus subtilis CH201 resulted in statistically significant improvements in standability, seedling height, root length, and yield as compared to check and as compared to the commercial products. Specifically, for standability, treatment of seed with the T2 formulation of Bacillus licheniformis CH200 and Bacillus subtilis CH201 at 70 g/100 kg and 100 g/100 kg seed provided at least about a 5% improvement over the commercial products QUALITY and AVICTA. For seedling development, treatment of seed with the T2 formulation of CH200 and CH201 strains at all rates resulted in an increase in height as compared to the check and resulted in a 9.9% increase in height as compared to the check at the highest rate. Further, the T2 formulation of CH200 and CH201 strains provided comparable results to QUALITY, and at the highest two rates outperformed AVICTA® by at least a 2.4% increase in height. For root mass development, the T2 formulation of CH200 and CH201 strains outperformed the check at all rates by at least 12.6 percent. Further, for the two highest rates, the T2 formulation of CH200 and CH201 strains outperformed both QUALITY and AVICTA® in root mass development by 4.2% and 2.6%, respectively. For yield, the T2 formulation of CH200 and CH201 strains outperformed the check at all rates by at least 8% percent and for the highest rate increased yield by 15.1%. All rates of the T2 formulation of CH200 and CH201 strains promoted higher yields than QUALITY. Further, the highest rate of the T2 formulation of CH200 and CH201 strains resulted in an increase in yield over AVICTA® of 3.4%. Thus, soybean seed treatment with the T2 formulation of CH200 and CH201 provides significant improvements in plant development and yield over a standard chemical treatment check and commercial nematicides under a range of low, medium, and high nematode pressure.

### Cotton

Six separate cotton field trials were performed in Brazil as described above under a range of low and medium nematode pressure. The data are shown below in Table 4, which shows the results of the average of the 6 trials on standability, seedling height, root length, and yield.

TABLE 4
Cotton field trials (average of 6 trials in Brazil) of seed treatment
with the T2 formulation of B. Licheniformis CH200 and B. Subtilis
CH201 as compared to commercial products QUALITY (a.i.
Thrichoderma) and nematicide AVICTA ® (a.i. abamectin).

| | | Seedling | Root | |
|---|---|---|---|---|
| | Standability | Height | length | |
| Seed Treatment | (plants/meter) | (cm) | (cm) | Yield |
| (g/100 kg) | 21 DAE | 35 DAE | 35 DAE | kg/hectare |
| Check | 9.2 | 36.6 | 18.6 | 3129 |
| T2 (80) | 9.5 | 39.0 | 20.2 | 3387 |
| T2 (160) | 9.3 | 39.5 | 19.7 | 3446 |
| T2 (200) | 8.8 | 39.1 | 19.7 | 3442 |
| T2 (300) | 9.4 | 40.0 | 20.4 | 3510 |
| QUALITY (200) | 9.5 | 36.8 | 20.4 | 3335 |
| AVICTA ® (300) | 9.6 | 39.1 | 20.0 | 3344 |

The results in Table 4 show that treating cotton seed with the T2 formulation of Bacillus licheniformis CH200 and Bacillus subtilis CH201 resulted in comparable or statistically significant improvements in standability, seedling height, root length, and yield as compared to check and as compared to the commercial products. Specifically, for seedling height, treatment of seed with the T2 formulation of CH200 and CH201 strains at all rates resulted in a statistical increase in height as compared to the check. At the highest application rate, application of the combination of strain provided a 9.3% and 8.7% increase in height as compared to the check and as compared to QUALITY, respectively. Further, at the highest rate, the T2 formulation of CH200 and CH201 strains outperformed AVICTA® by a 2.3% increase in height. For root development, treatment of seed with the T2 formulation of CH200 and CH201 strains at all rates resulted in a statistical increase in length as compared to the check and as compared to AVICTA® (a 9.8% and 2.0% increase in length, respectively, at the highest application rate). Further, treatment with the T2 formulation of CH200 and CH201 strains provided statistically similar results as compared to QUALITY at the highest rate. Treatment with of all rates of the T2 formulation of CH200 and CH201 strains promoted higher yields than the check and both commercial products QUALITY and AVICTA® (12.2% versus 5.2%, and 5.0%, respectively, for the highest rate). Thus, cotton seed treatment with the T2 formulation of CH200 and CH201 provides significant improvements over a standard chemical treatment check and commercial nematicides in plant development and yield under low and medium nematode pressure.

### Corn

Four separate corn field trials were performed in Brazil as described above under a range of medium and high nematode pressure. The data are shown below in Table 5, which shows the results of the average of the 4 trials on seedling height, root length, and yield.

TABLE 5
Corn field trials (average of 4 trials in Brazil) of seed treatment
with the T2 formulation of B. Licheniformis CH200 and B. Subtilis
CH201 as compared to commercial products QUALITY (a.i.
Thrichoderma) and AVICTA ® (a.i. abamectin).

| | Seedling Height | Root length | |
|---|---|---|---|
| Seed Treatment | (cm) | (cm) | Yield |
| (g/100 kg) | 35 DAE | 35 DAE | kg/hectare |
| Check | 92.0 | 22.6 | 4673 |
| T2 (40) | 91.6 | 24.8 | 4911 |
| T2 (80) | 90.9 | 23.5 | 4977 |
| T2 (100) | 92.8 | 25.8 | 5040 |
| T2 (120) | 92.8 | 25.9 | 5029 |
| QUALITY (100) | 88.8 | 25.9 | 5154 |
| AVICTA ® (70) | 89.9 | 24.5 | 5068 |

The results in Table 5 show that treating corn seed with the T2 formulation of Bacillus licheniformis CH200 and Bacillus subtilis CH201 resulted in statistically significant improvements in seedling and root development and yield as compared to check and as compared to the commercial products. For seedling height, treatment of seed with the T2 formulation of CH200 and CH201 strains provided improved results over the commercial products at all rates and, specifically, an improvement of 4.5% over QUALITY and an improvement of 3.2% over AVICTA at the highest rate. For root development, treatment with the T2 formulation of CH200 and CH201 strains outperformed the check at all rates. At the two highest rates, treatment with the T2 formulation of CH200 and CH201 strains outperformed the check and AVICTA® by at least 14.2% and 5.3%, respectively. Further, at the highest rate, treatment with the T2 formulation of CH200 and CH201 strains performed comparably to QUALITY. For yield, treatment with the T2 formulation of CH200 and CH201 strains outperformed the check at all rates and, specifically, the 100 g/100 kg rate increased yield by 7.9% over the check. In addition, the 100 g/100 kg rate of the T2 formulation of CH200 and CH201 strains provided yields statistically comparable to AVICTA. Thus, corn seed treatment with the T2 formulation of CH200 and CH201 provides significant improvements in plant development and yield over a standard chemical treatment check and provides comparable improvements in yield to commercial nematicides under medium and high nematode pressure.

### Dry Bean

Two separate dry bean field trials were performed in Brazil as described above under low and high nematode pressure. The data are shown below in Table 6, which shows the results of the average of the 2 trials on standability, seedling height, root length, and yield.

TABLE 6
Dry bean field trials (average of 2 trials in Brazil) of seed treatment
with the T2 formulation of B. Licheniformis CH200 and B. Subtilis
CH201 as compared to commercial products QUALITY (a.i.
Thrichoderma) and nematicide FURADAN (a.i. Carbofuran).*

| | | Seedling | Root | |
|---|---|---|---|---|
| | Standability | Height | length | |
| Seed Treatment | (plants/meter) | (cm) | (cm) | Yield |
| (g/100 kg) | 21 DAE | 35 DAE | 35 DAE | kg/hectare |
| Check | 13.1 | 43.7 | 14.3 | 1589 |
| T2 (40) | 14.5 | 51.4 | 21.2 | 1619 |
| T2 (80) | 13.9 | 50.8 | 21.2 | 1763 |
| T2 (100) | 13.4 | 49.4 | 19.0 | 1873 |
| T2 (120) | 14.3 | 49.6 | 16.8 | 1881 |
| QUALITY (100) | 13.6 | 48.1 | 15.1 | 1777 |
| FURADAN (3000) | 12.8 | 50.3 | 18.7 | 1887 |

The results in Table 6 show that treating soybean seed with the T2 formulation of Bacillus licheniformis CH200 and Bacillus subtilis CH201 resulted in statistically significant improvements in standability, seedling development, root mass, and yield as compared to check and as compared to the commercial products. Specifically, for standability, treatment of seed with the T2 formulation of CH200 and CH201 strains at the highest rate provided a 9.2%, 4.9%, and 11.7% improvement over the check, over QUALITY, and over FURADAN, respectively. For seedling development, treatment of seed with the T2 formulation of CH200 and CH201 strains at all rates resulted in an increase in height as compared to the check and as compared to QUALITY. In addition, the 80 kg/100 kg rate of the the T2 formulation of the CH200 and CH201 strains statistically outperformed FURADAN. For root length, all rates of treatment with the T2 formulation of CH200 and CH201 strains outperformed the check and QUALITY. The 3 lowest treatment rates of the T2 formulation of the CH200 and CH201 strains statistically outperformed or performed comparably to FURADAN. For yield, treatment with the T2 formulation of CH200 and CH201 strains outperformed the check at all rates and for the highest rate increased yield by 18.4%. Treatment with the two highest rates of the T2 formulation of CH200 and CH201 strains promoted higher yields than QUALITY (5.4% and 5.9%, respectively) and promoted comparable yields to FURADAN. Thus, seed treatment with the T2 formulation of CH200 and CH201 provides significant improvements in yield over a standard chemical treatment check and provides comparable to significant improvements in yield as compared to commercial nematicides under low and high nematode pressure.

### Example 3

### Seed Treatment with a Formulation of Bacillus Licheniformis CH200 and Bacillus Subtilis CH201 Increases Yield in Soybean in Field Trials Under High Nematode Infestation

Additional field trials were performed in Brazil with soybean seed treated with the formulation of CH200 and CH201 “T2” described in EXAMPLE 1 in areas of high nematode infestation. The results demonstrate that seed treatment with the T2 formulation of CH200 and CH201 strains significantly increases soybean yield, and the increase in yield is greater than that for the commercial product AVICTA® containing chemical active agent Abamectin.

In the field trials, all soybean seed was treated with MAXIM® XL containing Fludioxonil and Mefenoxam active chemical fungicidal ingredients and with chemical insecticide active agents Imidacloprid and Bifenthrin according to label (referred to herein as condition (1) base treatment (“BT”)). Condition (2) included further treatment of the seed with T2 at a rate of 70 g/100 kg seed. Condition (3) included further treatment of the seed with T2 at a rate of 100 g/100 kg seed. Condition (4) included further treatment of the seed with commercial product AVICTA® at 100 g/100 kg seed containing chemical active agent Abamectin for controlling nematodes. Field trials were conducted at two different locations with high nematode pressure and the average yield data are shown below in Table 7. The results demonstrate that seed treatment with the T2 formulation of CH200 and CH201 stains significantly increased yield relative to the base treatment at both rates (11.9% and 13.6%, respectively), and outperformed commercial product AVICTA®, which provided an increase in yield of 11.0%.

TABLE 7
Average soybean yield (kg/hectare) from field trials conducted
in Brazil at two different locations under high nematode pressure.

| | | Soybean Yield | Percent |
|---|---|---|---|
| Treatment | | (kg/hectare) | Increase |
| 1 | BT | 2532 | 0 |
| 2 | BT + T2 (70 g/100 kg seed) | 2834 | 11.9 |
| 3 | BT + T2 (100 g/100 kg seed) | 2876 | 13.6 |
| 4 | BT + AVICTA ® (100 g/100 kg seed) | 2810 | 11.0 |

### Example 4

### Greenhouse Studies in Soybean Show Seed Treatment with Formulation of the of Bacillus Licheniformis CH200 and Bacillus Subtilis CH201 Reduces Nematodes in Infested Soil

Greenhouse studies of soybean demonstrate that the T2 formulation of the B. licheniformis CH200 and B. subtilis CH201 strains outperforms commercially available products for controlling nematodes. The following experimental data are provided to show that application of the T2 formulation of the CH200 and CH201 strains to soybean infected with various types of nematodes can outperform commercially available products based on both chemical and biological active agents.

In the experiment, soybean seeds were planted in a single pot infected with 600 Pratylenchus brachyurus nematodes per pot or 1000-2000 Meloidogyne javanica nematodes per pot. In this experiment, all seed was treated with MAXIM® XL (SYNGENTA CROP PROTECTION, INC) containing Fludioxonil and Mefenoxam active chemical fungicidal ingredients according to label (referred to as condition (1) Base treatment (“BT”)). Condition (2) included further treatment of the seed with T2 from EXAMPLE 1 at a rate of 100 g/100 kg seed. Condition (3) included further treatment of the seed with commercial product AVICTA® (SYNGENTA, INC) at 100 g/100 kg seed containing chemical active agent Abamectin for controlling nematodes. Condition (4) included further treatment of the seed with commercial product NEMAT (SYNGENTA, INC) at 100 g/100 kg seed containing fungal active agent Paecilomyces lilacinus (7.5×10⁹ CFU/g) for controlling nematodes. The data for initial penetration of Meloidogyne javanica nematodes 21 days after inoculation (DAI) are shown in Table 8 below. The results demonstrate that the T2 formulation of the CH200 and CH201 stains performs statistically as well in controlling nematode penetration as commercial chemical product AVICTA, and outperforms commercial product NEMAT containing biological active agent Paecilomyces lilacinus.

TABLE 8
Initial penetration of Meloidogyne javanica 21 days after inoculation.

| Treatment | | Initial penetration, 21 DAI |
|---|---|---|
| 1 | BT | 109 |
| 2 | BT + T2 (100 g/100 kg seed) | 36 |
| 3 | BT + AVICTA ® (100 g/100 kg seed) | 27 |
| 4 | BT + NEMAT (100 g/100 kg seed) | 50 |

The number of juvenile Meloidogyne javanica nematodes per 5 gram roots at 40 days after the inoculation (DAI) was examined and the results are shown in Table 9 below. The results demonstrate that the T2 formulation of the CH200 and CH201 stains performs statistically as well in controlling nematode juveniles as commercial chemical product AVICTA®, and outperforms commercial product NEMAT containing biological active agent Paecilomyces lilacinus.

TABLE 9
Number of juvenile Meloidogyne javanica nematodes per
5 gram roots at 40 days after inoculation (DAI).

| Treatment | | Juveniles on roots, 40 DAI |
|---|---|---|
| 1 | BT | 1695 |
| 2 | BT + T2 (100 g/100 kg seed) | 437 |
| 3 | BT + AVICTA ® (100 g/100 kg seed) | 366 |
| 4 | BT + NEMAT (100 g/100 kg seed) | 776 |

The number of Meloidogyne javanica nematode eggs per 5 gram roots at 60 DAI was examined and the results are shown in Table 10 below. The results demonstrate that the T2 formulation of the CH200 and CH201 stains statistically outperforms chemical product AVICTA® in controlling nematode eggs on roots. In this case, the T2 formulation of the CH200 and CH201 stains does not perform as well as the commercial product NEMAT containing biological active agent Paecilomyces lilacinus. The P. lilacinus strain is known for its ability to kill nematode eggs.

TABLE 10
Number of eggs of Meloidogyne javanica per 5 gram
roots at 60 days after inoculation (DAI).

| Treatment | | Eggs on roots, 60 DAI |
|---|---|---|
| 1 | BT | 6162 |
| 2 | BT + T2 (100 g/100 kg seed) | 3167 |
| 3 | BT + AVICTA ® (100 g/100 kg seed) | 4001 |
| 4 | BT + NEMAT (100 g/100 kg seed) | 839 |

The reproduction factor (RF—Final nematode population divided by Initial nematode population) of Pratylenchus brachyurus nematodes at 60 and 90 DAI was examined and the results are shown in Table 11 below. The results demonstrate that the T2 formulation of the CH200 and CH201 stains statistically outperforms chemical product AVICTA in controlling nematode reproduction at both 60 and 90 days DAI. In addition, the T2 formulation of the CH200 and CH201 stains outperforms the commercial product NEMAT containing biological active agent Paecilomyces lilacinus at 60 DAI and performs as well as this product at 90 DAI. These data show that the T2 formulation of the CH200 and CH201 stains can control nematodes better than the commercial chemical nematicide active agent and can control nematodes as well or even outperform the commercial nematicide product NEMAT containing Paecilomyces lilacinus.

TABLE 11
Reproduction factor (RF - Final nematode population divided
by Initial nematode population) of Pratylenchus brachyurus
at 60 and 90 days after inoculation (DAI).

| | | RF | |
|---|---|---|---|
| Treatment | | 60 DAI | 90 DAI |
| 1 | BT | 2.9 | 4.9 |
| 2 | BT + CH200/CH201 (100 g/100 kg seed) | 1.1 | 2.9 |
| 3 | BT + AVICTA ® (100 g/100 kg seed) | 2.4 | 10.5 |
| 4 | BT + NEMAT (100 g/100 kg seed) | 3.5 | 2.3 |

1. A wettable powder composition for plant seed treatment or in-furrow application to control plant pathogenic nematodes, the composition comprising by weight percent:
5-20% of a culture of not less than about 1×10¹¹ CFU/g of Bacillus licheniformis CH200 deposited as DSM 17236, or a mutant thereof having all the identifying characteristics thereof;
5-20% of a culture of not less than about 1×10¹¹ CFU/g of Bacillus subtilis CH201 deposited as DSM 17231, or a mutant thereof having all the identifying characteristics thereof;
5-15% of maltodextrin;
35-45% of calcium carbonate; and
5-15% of silica.

2. A composition for plant seed treatment or in-furrow application to control plant pathogenic nematodes, wherein the composition is in the form of a dust, a dry wettable powder, a spreadable granule, or a dry wettable granule, the composition comprising by weight percent:
7-40% of a culture of not less than about 1×10¹¹ CFU/g of Bacillus licheniformis CH200 deposited as DSM 17236, or a mutant thereof having all the identifying characteristics thereof;
7-40% of a culture of not less than about 1×10¹¹ CFU/g of Bacillus subtilis CH201 deposited as DSM 17231, or a mutant thereof having all the identifying characteristics thereof;
wherein the composition comprises a solid carrier selected from the group consisting of mono- or di-saccharides, oligo- or poly-saccharides, talc, titanium dioxide, pyrophyllite clay, attapulgite clay, kieselguhr, silica, limestone, bentonite, calcium montmorillonite, sodium, potassium, magnesium, calcium or ammonium salts of acetate, carbonate, chloride, citrate, phosphate, or sulfate, cottonseed husks, wheat flour, soybean flour, pumice, wood flour, ground nut shells, lignin, yeast extracts, fish meal, and mixtures thereof.

3. The composition of claim 2 wherein the solid carrier comprises maltodextrin, silica, calcium carbonate, or any mixtures thereof.

4. The composition of claim 3 comprising 5-15% of maltodextrin.

5. The composition of any of claim 1, 2, 3 or 4, wherein plant seed treatment or in furrow application of the composition benefits plant growth and yield.

6. An article of manufacture comprising a plant seed treated with the composition of any of claim 1, 2, 3 or 4.

7. The article of manufacture of claim 6, wherein the plant seed comprises soybean, dry bean, cotton, or corn.

8. The article of manufacture of claim 6 or claim 7, wherein the plant seed is further treated with one or a combination of a microbial, a biological, or a chemical insecticide, fungicide, nematicide, bacteriocide, herbicide, plant extract, plant growth regulator, or fertilizer present in an amount suitable to benefit plant growth and/or to confer protection against a pathogenic infection in the plant.

9. The article of manufacture of claim 8, wherein the chemical fungicide is one or a combination of mefenoxam, fluopyram, chlorothalonil, thiophanate-methyl, fludioxonil, metalaxyl, or sedaxane.

10. The article of manufacture of claim 8, wherein the chemical insecticide is one or a combination of imidacloprid, bifenthrin, thiamethoxam, pyrethroids, tefluthrin, zeta-cypermethrin, organophosphates, chlorethoxyphos, chlorpyrifos, tebupirimphos, cyfluthrin, fiproles, fipronil, nicotinoids, or clothianidin.

11. The article of manufacture of claim 8, wherein the chemical insecticide comprises chlorantraniliprole, cyantraniliprole, cyclaniliprole or indoxacarb, optionally in combination with imidacloprid, bifenthrin, thiamethoxam, pyrethroids, tefluthrin, zeta-cypermethrin, organophosphates, chlorethoxyphos, chlorpyrifos, tebupirimphos, cyfluthrin, fiproles, fipronil, nicotinoids, or clothianidin.

12. A method for controlling plant pathogenic nematodes, the method comprising:
delivering to seed of a plant, roots of a plant, or soil surrounding a plant a composition of any of claim 1, 2, 3 or 4,
wherein the composition delivered to seed of the plant, roots of the plant, or soil surrounding the plant improves the plant growth and/or yield in the presence of a plant pathogenic nematode, improves the plant resistance to a plant pathogenic nematode, or reduces the plant infection by a plant pathogenic nematode, and combinations thereof.

13. A method for controlling plant pathogenic nematodes, the method comprising:
delivering to seed of a plant, roots of a plant, or soil surrounding a plant a liquid composition in the form of a suspension concentrate or an oil dispersion comprising by weight percent:
0.5-40% of a biologically pure culture of not less than about 1×10¹¹ CFU/g Bacillus licheniformis CH200 deposited as DSM 17236, or a mutant thereof having all the identifying characteristics thereof;
0.5-40% of a biologically pure culture of not less than about 1×10¹¹ CFU/g Bacillus subtilis CH201 deposited as DSM 17231, or a mutant thereof having all the identifying characteristics thereof;
a liquid carrier;
a surface active agent; and
an adjuvant selected from the group consisting of preservatives, biocides or biostatic agents, thickeners, antifoams, and antifreezes, viscosity modifiers, biocides or biostatic agents, crystallization inhibitors, suspending agents, dyes, anti-oxidants, foaming agents, light absorbers, mixing auxiliaries, complexing agents, neutralizing or pH-modifying substances and buffers, corrosion inhibitors, fragrances, wetting agents, take-up enhancers, micronutrients, plasticizers, glidants, lubricants, and dispersants;
wherein the composition is optionally further diluted with water; and
wherein the composition delivered to seed of the plant, roots of the plant, or soil surrounding the plant improves the plant growth and/or yield in the presence of a plant pathogenic nematode, improves the plant resistance to a plant pathogenic nematode, or reduces the plant infection by a plant pathogenic nematode, and combinations thereof.

14. The method of claim 13 wherein the liquid composition is a suspension concentrate comprising water and at least one surface active agent, and one or more additional adjuvants selected from thickeners, solvents, preservatives, antifreeze agents, and antifoam agents.

15. The method of claim 14 wherein the liquid composition is a suspension concentrate comprising from 1 to 10 weight % of the Bacillus licheniformis culture or a mutant thereof having all the identifying characteristics thereof; from 1 to 10 weight % of the Bacillus subtilis culture or a mutant thereof having all the identifying characteristics thereof; 1 to 5 weight % of one or more surface active agent; and at least one thickener, solvent, preservative, antifreeze agent, or antifoam agent each independently comprising up to about 1 weight % of the composition.

16. The method of any of claim 12, 13, 14 or 15, wherein the plant pathogenic nematode comprises a Meloidogyne spp. or a Pratylenchus.

17. The method of any of claim 12, 13, 14, 15 or 16, wherein the plant comprises soybean, cotton, dry bean, corn, sugarcane, coffee, or potato.

18. The method of any of claim 12, 13, 14, 15, 16 or 17, wherein delivery of the composition improves plant yield in the presence of a plant nematode pathogen.

19. The method of any of claim 12, 13, 14, 15, 16, 17 or 18, further comprising delivering to seed of the plant, roots of the plant, or soil surrounding the plant one or a combination of a microbial, a biological, or a chemical insecticide, fungicide, nematicide, bacteriocide, herbicide, plant extract, plant growth regulator, or fertilizer present in an amount suitable to benefit plant growth and/or to confer protection against a pathogenic infection in the plant.

20. The method of claim 19, wherein the chemical fungicide is one or a combination of mefenoxam, fluopyram, chlorothalonil, thiophanate-methyl, fludioxonil, metalaxyl, or sedaxane.

21. The method of claim 19, wherein the chemical insecticide is one or a combination of imidacloprid, bifenthrin, thiamethoxam, pyrethroids, tefluthrin, zeta-cypermethrin, organophosphates, chlorethoxyphos, chlorpyrifos, tebupirimphos, cyfluthrin, fiproles, fipronil, nicotinoids, or clothianidin.

22. The method of claim 19, wherein the chemical insecticide comprises chlorantraniliprole, cyantraniliprole, cyclaniliprole or indoxacarb, optionally in combination with imidacloprid, bifenthrin, thiamethoxam, pyrethroids, tefluthrin, zeta-cypermethrin, organophosphates, chlorethoxyphos, chlorpyrifos, tebupirimphos, cyfluthrin, fiproles, fipronil, nicotinoids, or clothianidin.

23. A process for preparing a composition according to any of claim 1, 3 or 4, the process comprising mixing, in a dry or powder form:
a biologically pure culture of not less than about 1×10¹¹ CFU/g Bacillus licheniformis CH200 deposited as DSM 17236, or a mutant thereof having all the identifying characteristics thereof;
a biologically pure culture of not less than about 1×10¹¹ CFU/g Bacillus subtilis CH201 deposited as DSM 17231, or a mutant thereof having all the identifying characteristics thereof;
maltodextrin;
calcium carbonate; and
silica,
wherein the mixing is in a ratio to effect the weight percent of the composition according to claim 1, 3 or 4.
